(19) United States
(12) Patent Application Publication
(10) Pub. No.: US 2007/0078435 A1
(43) Pub. Date: Apr. 5, 2007

(54) Tissue augmentation methods using a medical injection apparatus

(76) Inventors: Corbett Stone, San Diego, CA (US); Russell Anderson, San Diego, CA (US)

Correspondence Address: HELLER EHRMAN LLP, 4350 LA JOLLA VILLAGE DRIVE #700, 7TH FLOOR, SAN DIEGO, CA, 92122, US

(21) Appl. No.: 11/454,540

(22) Filed: Jun. 16, 2006

Related U.S. Application Data
(63) Continuation-in-part of application No. 10/714,154, filed on Nov. 14, 2003, now abandoned.
Application No. 10/714,154 is a continuation of application No. 10/172,773, filed on Jun. 14, 2002, now Pat. No. 6,666,848.
(60) Provisional application No. 60/298,310, filed on Jun. 14, 2001.
(60) Provisional application No. 60/298,620, filed on Jun. 14, 2001.

(51) Int. Cl. A61M 31/00

(52) U.S. Cl. 604/507; 604/272; 604/164.01

(57) ABSTRACT

An injection apparatus that includes components that facilitate injection of relatively viscous materials into a subject is provided as well as methods of use. An injection apparatus may include a transition-bore needle apparatus, which has a proximal end, a distal end, and a lumen extending from the proximal end to the distal end, in which the diameter of the proximal end is greater than the diameter of the distal end. An injection apparatus may include a hand-held injection facilitation apparatus, which may be coupled to a syringe. The hand-held injection facilitation apparatus can include a pivot arm and a body with a rod disposed within the body and coupled to the pivot arm. Movement of the pivot arm results in a proximal or distal movement of the rod within the body to effectively cause material to be expelled from the syringe. An injection apparatus may include a transition-bore needle apparatus and a hand-held injection facilitation apparatus in combination.

### CROSS-REFERENCE TO RELATED APPLICATIONS

This application is a continuation in part of application Ser. No. 10/714,154, which is a continuation of application Ser. No. 10/172,773, now U.S. Pat. No. 6,666,848, which claims the benefit of U.S. Provisional Application No. 60/298,310, entitled INJECTION FACILITATION APPARATUS, and filed Jun. 14, 2001; and U.S. Provisional Application No. 60/298,620, entitled TRANSITION-BORE NEEDLE APPARATUS, and filed Jun. 14, 2001, the entire contents of which are hereby incorporated by reference.

### BACKGROUND OF THE INVENTION

1. Field of the Invention

The present invention relates generally to medical devices and, more particularly, to injection apparati that facilitate and improve the ability to pass viscous materials through lumens of small aperture and methods of use for tissue augmentation.

2. Description of Related Art

The term “stress urinary incontinence” refers to a functionally insufficient urinary tract of a subject. In a subject having this condition, the tissue relaxation of the sphincter mechanism, located at the urinary outflow of the bladder into the urethra, can cause a loss of bladder control. Cystoscopes are typically used to study the urethra and bladder and to evaluate, for example, a subject's urinary incontinence condition. A typical cystoscope may comprise a tubular instrument equipped with, for example, a visual channel and a working channel, and constructed to be inserted through the urethra for viewing of the urethra and bladder.

Treatment of a urinary incontinence condition may comprise the injection of a filler material, such as collagen, into and adjacent to the urinary sphincter muscle at the bladder neck, to thereby bulk up the tissue and assist in the adequate closure of the urinary sphincter.

Acid reflux is a digestive disorder which similarly involves the tissue relaxation of a sphincter mechanism. In the case of acid reflux, which is commonly known as gastroesophageal reflux disease (GERD) or heartburn, the lower esophageal sphincter connecting the esophagus to the stomach begins to malfunction.

During proper operation of the lower esophageal sphincter, the lower esophageal sphincter opens to allow food to pass into the stomach and closes to prevent food and acidic stomach fluids from flowing back up into the esophagus. Gastroesophageal reflux occurs when the lower esophageal sphincter is weak or relaxes inappropriately, allowing the stomach's contents to retrograde or flow up into the esophagus.

This retrograde flow of gastric contents back into the esophagus, through what should be a one-way valve into the stomach, can damage the esophagus. More particularly, the contents of the stomach are very acidic; and the lining of the stomach is specially designed to cope with the lower pH contents. The esophagus, on the other hand, is not suited for such exposure to highly acidic materials. Thus, when acid retrogrades from the stomach into the esophageal tissues, irritation and inflammation will often result to these tissues.

The severity of tissue damage which can result from gastroesophageal reflux disease can depend on factors such as the dysfunctional level of the lower esophageal sphincter, the type and amount of fluid brought up from the stomach, and the neutralizing effect of the subject's saliva.

Another factor, which may affect the severity of a particular gastroesophageal reflux disorder, is the subject's esophageal motility. Lack of esophageal motility can occur through either of two mechanisms. When incomplete emptying of the esophagus into the stomach after ingestion of liquids or solids occurs, the motility of the esophagus can be said to be affected, resulting in esophageal reflux. Also, esophageal reflux can occur when small amounts of gastric contents, which may be refluxed into the lower esophagus, are not rapidly emptied back into the stomach. Delays in the emptying of this material, caused by an esophageal motility disorder, for example, can lead to irritation of the esophageal mucosa and possibly to the sensation of heartburn or the development of esophagitis.

Embolization is the selective blockage of one or more blood vessels supplying a diseased vascular structure or diseased tissue while simultaneously preserving the blood supply to surrounding normal vascular structure or tissue. For example, uterine fibroid embolization (UFE) is the process of occluding the vascular blood supply to uterine fibroids to reduce fibroid size and alleviate associated symptoms, including bleeding, pain, and disfigurement. Tumor embolization is the process of selectively blocking blood supply to a tumor. Tumor embolization can also be the process of selectively blocking blood supply away from the tumor in order to, for example, prevent or limit metastasis.

Cartilage is the tough, elastic, fibrous connective tissue that is a major constituent of embryonic and young vertebrate skeletons, is converted largely to bone with maturation, and is found in various parts of the adult body, such as the joints, outer ear, and larynx. Cartilage can play an important role, for example, in joint mobility. For example, at one time knee menisci were once thought to be expendable components of the knee, but are now known to be involved in joint stability, load sharing and transmission, shock absorption, and nutrition and lubrication of the articular cartilage. Consequently, cartilage damage due to age or trauma can require therapeutic intervention.

Spinal disks comprise a central region called the nucleus pulposus surrounded by a second region known as the annulus fibrosis. The annulus fibrosis portion comprises collagen fibers that may weaken, rupture, or tear, leading to compromised annular confinement of the nucleus and producing disk bulges, herniations and other disk pathologies. The major causes of persistent, often disabling, back pain are disruption of the spinal disk annulus fibrosis, chronic inflammation of the spinal disk (e.g., herniation), or relative instability of the vertebral bodies surrounding a given spinal disk, such as the instability that often occurs due to a degenerative disease.

Sphincter muscles are ringlike muscles that normally maintain constriction of a body passage or orifice and that relax as required by normal physiological functioning. Flaccidity or abnormal functioning due to, for example, age or disease, can result in debilitating and embarrassing conditions. For example, dysfunction of the anal sphincter can result in fecal incontinence, dysfunction of the bladder sphincter can result in urinary incontinence, and dysfunction of the pyloric sphincter can result in enhanced gastric emptying.

Muscle is tissue consisting predominantly of contractile cells and classified as skeletal, cardiac, or smooth, the last lacking transverse striations characteristic of the first two. Muscle tears can excruciatingly painful and limit functionality. Defects in the heart muscle can be life threatening.

A tendon is a band of tough, inelastic fibrous tissue that connects a muscle with its bony attachment. A ligament is sheet or band of tough, fibrous tissue connecting bones or cartilages at a joint or supporting an organ. Disruption or tearing of a tendon or ligament can be excruciatingly painful and limit functionality of the connected structure. For example, the anterior cruciate ligament (ACL) is essential for guiding the tibia (shinbone) in a normal path along the end of the femur (thighbone) and maintaining stability of the knee joint. When this ligament is torn or ruptured, the joint loses stability and further destruction of the articular and meniscal cartilage results, i.e. degenerative arthritis. The most common reconstruction of the ACL involves the use of patellar tendon and hamstring grafts, with cadaver grafts representing a third option. Suturing is sometimes an option, but 50% of these procedures are reported to fail because of the strain placed on the knee. All of these methods are expensive, require exceptional surgical skill, require long recovery times and are sometimes unsuccessful. Various tools and instruments have been used for treating the above anatomic structures. For example, gastroscopes are typically used to study the esophagus and to evaluate, for example, a subject's acid reflux condition. A gastroscope typically comprises a flexible, lighted instrument that is inserted through the mouth and esophagus to view the stomach. Similarly, a cystoscope is typically inserted through a subject's urethra to facilitate evaluation of, for example, a urinary incontinence condition.

Tissue augmentation of the above-mentioned anatomic structures may include one or more injections of a viscous material, such as collagen, into the vicinity of the associated anatomic structure. These injection procedures typically involve elongate catheters for the delivery of viscous materials through the body passages and to the target sites of injection. The force required to deliver a viscous material through the delivery lumen of an elongate catheter will naturally increase as the length of the elongate catheter increases. Moreover, the types of elongate catheters used with these surgical procedures will typically have delivery lumens of relatively small cross-sectional areas, thus further augmenting the force required to deliver the viscous material through the length of the elongate catheter.

To compensate for the greater required force, it would be desirable to form the elongate catheter to have a lumen with a relatively large cross-sectional area to facilitate flow of the viscous material therethrough. Another design criterion is that the diameter of the needle tip should be relatively small to reduce tissue trauma at the injection site, to increase precision in some instances, and to reduce subject discomfort.

In order to meet the objectives of both a relatively large delivery lumen and a relatively small needle tip, a juncture must be formed at some point along the length of the needle to transition the needle diameter from a relatively large size to a relatively small size. If the transition point is abrupt or too great in magnitude, optimal flow of the viscous material through the needle may be inhibited.

Thus, there is a need for an apparatus and methods that can deliver viscous material through a body passage of a subject to a target site of injection. The present invention satisfies these needs and provides further advantages.

### SUMMARY OF THE INVENTION

An injection apparatus, as disclosed herein, may comprise a transition-bore needle apparatus to optimize the flow of viscous material from the injection apparatus. An injection apparatus, as disclosed herein, may comprise a hand-held injection facilitation apparatus which reduces the effort required to displace viscous material from the injection apparatus. The hand-held injection facilitation apparatus is structured to cooperatively interact with a syringe to cause displacement of viscous material from the syringe. An injection apparatus may comprise a combination of a transition-bore needle apparatus, and a hand-held injection facilitation apparatus, disclosed herein.

A transition-bore needle apparatus is provided to optimize the flow of a viscous material through a decreasing-diameter lumen of a needle. The transition-bore needle apparatus comprises a proximal end and a distal end, and the lumen extends from the proximal end of the transition-bore needle apparatus to the distal end of the transition-bore needle apparatus. A diameter at a proximal portion of the transition-bore needle apparatus is greater than a diameter at a distal portion of the transition-bore needle apparatus.

In accordance with one aspect of the present invention, the proximal portion of the transition-bore needle apparatus comprises a first needle having a first diameter, and the distal portion of the transition-bore needle apparatus comprises a second needle having a second diameter. The first diameter is greater than the second diameter. The first needle comprises a proximal end, a distal end, and a first lumen extending through the first needle from the proximal end to the distal end, and the second needle similarly comprises a proximal end, a distal end, and a second lumen extending through the second needle from the proximal end of the second needle to the distal end of the second needle.

The lumen of the transition-bore needle apparatus comprises both a portion of the first lumen of the first needle and a portion of the second lumen of the second needle. A juncture thus exists within the lumen of the transition-bore needle apparatus, where the diameter thereof transitions from the first diameter to the second diameter. At this juncture, the proximal end of the first needle terminates within the second lumen. In accordance with an aspect of the present invention, the proximal end of the first needle is beveled to improve a flow of viscous material through the lumen of the transition-bore needle apparatus. In accordance with another aspect of the present invention, the proximal end of the first needle is chamfered to improve a flow of viscous material through the lumen of the transition-bore needle apparatus. According to yet another aspect of the present invention, the proximal end of the first needle is both chamfered and beveled to improve a flow of viscous material through the lumen of the transition-bore needle apparatus.

The transition-bore needle apparatus of the facilitates the injection of viscous filler material by optimizing a flow of the viscous material through the lumen of the transition-bore needle apparatus. The transition-bore needle apparatus may be used in conjunction with surgical instruments, such as endoscopes, proctoscopes, cystoscopes, uroscopes, laproscopes, bronchoscopes, otoscopes, laryngoscopes, arthroscopes, angioscopes and gastroscopes, to aid in intraluminal injections of materials into body tissues within body lumens. When the body lumen comprises an esophagus, the gastroscope is inserted through the esophagus into a vicinity of the lower esophageal sphincter, and a long needle is used to inject a filler material into and adjacent to the lower esophageal sphincter tissues for the treatment of acid reflux. When the body lumen comprises a female urethra, the cystoscope is inserted through the urethra to the urinary sphincter adjacent to the bladder neck, and a long needle is used to inject a filler material into and adjacent to the urinary sphincter tissues for the treatment of stress urinary incontinence. The filler material may also be injected, for example, along a greater length of the urethra.

The injection apparatus disclosed herein thus facilitates the injection of viscous filler materials, and may provide for increased speed, accuracy and efficiency in dispensing such materials. In some embodiments, an injection apparatus where portions of the injection apparatus are made partly or entirely of liquid crystal polymer (LCP) are provided.

The injection of bulking agents into the respective tissues of body sphincters helps fortify the respective tissue structures and re-establish normal sphincter control. The transition-bore needle apparatus of the injection apparatus and associated methods of operation disclosed herein may be configured for and used on other body passages and tissues (e.g., wrinkles) as well in modified embodiments. A depth indicator can also be incorporated with the injector to allow a user to easily monitor and ensure that the desired injection depth is maintained during the procedure.

The transition bore needle apparatus of the injection apparatus and associated methods of operation can further include the use of external visualization methods such as radiography, fluoroscopy, computerized tomography, or ultrasound. Compatible image contrast reagents known to those skilled in the art can be used in conjunction with the various embodiments of the invention to improve guidance of the needle tip to the desired injection site.

An injection apparatus may comprise a hand-held injection facilitation apparatus to increase the precision of dispensing of a material from a syringe. The injection facilitation apparatus may be used in conjunction with surgical instruments, such as endoscopes, proctoscopes, cystoscopes, uroscopes, laproscopes, bronchoscopes, otoscopes, laryngoscopes, arthroscopes, angioscopes and gastroscopes for intraluminal injections of materials into body tissues, and to visualize tissue within a body lumen. For example, when the body lumen comprises an esophagus, a gastroscope can be inserted through the esophagus into a vicinity of the lower esophageal sphincter, and a long needle can be used to inject a filler material into or adjacent, or into and adjacent, to the lower esophageal sphincter tissues for the treatment of acid reflux. When the body lumen comprises a female urethra, a cystoscope can be inserted through the urethra into the urinary sphincter adjacent to the bladder neck, and a long needle is used to inject a filler material into or adjacent, or into and adjacent, to the urinary sphincter muscle tissues for the treatment of stress urinary incontinence. The filler material can also be injected, for example, along the entire length of the urethra.

The hand-held injection facilitation apparatus of the injection apparatus facilitates the injection of the viscous filler materials, and provides for increased accuracy in dispensing such materials. The injection of a tissue bulking agents into the respective tissues of body sphincters helps fortify the respective tissue structures and re-establish normal bladder sphincter control. The apparatus of the present invention and associated methods of operation disclosed herein may be configured for and used on other body passages or lumens as well in modified embodiments.

In accordance with one aspect of the present invention, an injection facilitation apparatus is constructed for use in conjunction with a stainless steel needle tip catheter that can be introduced into a subject's body lumen. For example, an injection facilitation apparatus is constructed for use in conjunction with a stainless steel needle tip catheter that can be introduced into a subject's urethra or esophagus or other targeted injection site to provide tissue augmentation to such structures such as in a treatment for urinary incontinence or gastro-esophageal reflux, respectively. The treatment for gastro-esophageal reflux disease can be fashioned to increase the strength or the length of the lower esophageal sphincter (LES) by depositing a viscous material around the lower esophageal sphincter. The suspension can be injected via a syringe and needle directly into the specific areas where the viscous agent is desired. A principal use of the exemplary embodiment is to accurately dispense the viscous material to thereby alter the physiological architecture of the subject's sphincter and adjacent tissues. Thus the bio-mechanical characteristics of the sphincter and surrounding tissues are altered to alleviate urinary incontinence and gastro-esophageal reflux.

In another aspect of the disclosure, methods are provided for tissue augmentation of anatomic structures within a body lumen. Included are methods for vascular embolization, tumor metastasis, cartilage augmentation, muscle augmentation, tendon augmentation, anal sphincter muscle augmentation, bladder sphincter muscle augmentation, pyloric sphincter muscle augmentation, and vertebral disc augmentation. Methods are also provided for delivery of tissue augmentation materials that include autologous fluids or tissues. Methods are also provided for delivery of tissue augmentation materials that include stem cells or other extracellular materials, or stem cells and other extracellular materials.

The subjects and objects of this disclosure relate to novel methods and instruments for facilitating the controlled dispensing of viscous material in the interior of the body, including but not limited to soft tissues, and lumen structures (e.g., esophagus, urethra).

The present invention, together with additional features and advantages thereof, may best be understood by reference to the following description taken in connection with the accompanying illustrative drawings.

### BRIEF DESCRIPTION OF THE DRAWINGS

FIG. 1 illustrates a transition-bore needle apparatus for use in the treatment of urinary incontinence in accordance with the present invention;

FIG. 1A is a cross-sectional view of a proximal end of a distal needle with edges beveled at about a 45 degree angle from the longitudinal axis of the distal needle;

FIG. 1B is a cross-sectional view of a proximal end of a distal needle with edges chamfered at about a 45 degree angle from the longitudinal axis of the distal needle;

FIG. 2 illustrates a transition-bore needle apparatus for used in the treatment of gastro-esophageal reflux disease in accordance with the present invention;

FIG. 2A is a cross-sectional view of a proximal end of a needle with edges chamfered at about a 30 degree angle from the longitudinal axis of the needle;

FIG. 2B is a cross-sectional view of a proximal end of a needle with edges beveled at about a 30 degree angle from the longitudinal axis of the needle;

FIG. 3 illustrates an injection facilitation apparatus with an attached syringe for use with a transition-bore needle apparatus;

FIG. 4 shows a part cross-sectional view of the injection facilitation apparatus, showing the housing and its internal components;

FIG. 5 is a part cross-sectional view of the injection facilitation apparatus wherein the handle is in a neutral position with no external force being applied to the handle;

FIG. 5A is a side-elevational view of the housing of the injection facilitation apparatus of FIG. 5, taken along the line A-A of FIG. 5;

FIG. 5B is a side-elevational view of an internal end of the pivot arm of the injection facilitation apparatus of FIG. 5, taken along the line B-B of FIG. 5;

FIG. 6 is a part cross-sectional view of the injection facilitation apparatus of FIG. 5 after an initial application of external force has been applied to the handle;

FIG. 7 illustrates the injection facilitation apparatus of FIG. 5 at a time of maximum application of external force to the handle; and

FIG. 8 illustrates a gastroscope used in the urethra to treat urinary incontinence in accordance with the present invention.

### DETAILED DESCRIPTION OF THE PRESENTLY PREFERRED EMBODIMENTS

An injection apparatus that facilitates displacement of viscous materials from a catheter may comprise a transition-bore needle apparatus, a hand-held injection facilitation apparatus, or a combination thereof. The transition-bore needle apparatus and the hand-held injection facilitation apparatus, as disclosed herein, can reduce the effort required by a person to displace the viscous material from the injection apparatus. In general, the transition-bore needle apparatus can reduce the effort required to expel material from a catheter by graduating changes of the internal diameter of the lumens of catheters. The hand-held injection facilitation apparatus can reduce the effort required to expel material from a catheter by permitting a person to control the longitudinal displacement of a syringe plunger using a gripping action of the person's hand as compared to a digit action between the person's thumb and fingers, as is conventionally practiced.

In one embodiment, any or all components are made from standard material, such as surgical grade stainless steel. Any or all of the components can comprise material that can be resistant to absorbing filler material or constituent part of the filler material's composition. Such component material can also or alternatively be resistant to leaching of materials from the apparatus component parts. For example, any or all of the component parts of the medical injection apparatus can comprise liquid crystal polymer (LCP) compositions, particularly where the component part comes into contact with the filler material, such as disclosed in co-owned patent application entitled “LCP Syringes and Containers”, filed Jun. 16, 2006, which cites priority to “Liquid Crystal Polymer Syringe”, U.S. Provisional Patent Application No. 60/691,506, filed Jun. 16, 2005, both of which are herein incorporated by reference. The absorption or leaching resistant material can either coat surfaces of other materials, such as surgical grade stainless steel or glass, or can comprise a component or a sleeve for the component part.

The transition-bore needle apparatus of the invention facilitates the injection of viscous filler material by optimizing a flow of the viscous material through the lumen of the transition-bore needle apparatus. Said apparatus of the invention can also be used with non-viscous filler material. The transition-bore needle apparatus may be used in conjunction with surgical instruments, such as endoscopes, proctoscopes, cystoscopes, uroscopes, laproscopes, bronchoscopes, otoscopes, laryngoscopes, arthroscopes, angioscopes and gastroscopes, to aid in intraluminal injections of materials into body tissues within body lumens.

The transition bore needle apparatus of the injection apparatus and associated methods of operation may further include the use of external visualization methods such as radiography, fluoroscopy, computerized tomography, or ultrasound. Compatible image contrast reagents known to those skilled in the art can be used in conjunction with the various embodiments of the invention to improve guidance of the needle tip to the desired injection site.

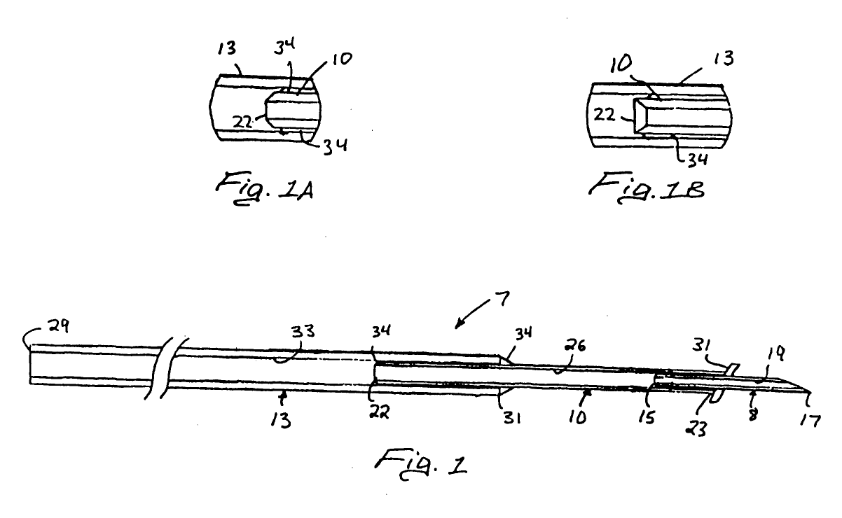

Referring more particularly to the drawings, FIG. 1 illustrates a transition-bore needle apparatus 7 adapted for use in applications such as urethral injections, and having a distal needle 8, an intermediate tube 10, and a proximal tube 13. Transition-bore needle apparatus 7 is an element of an injection apparatus, as disclosed herein. It is to be understood that, as used herein, the term “proximal” means the end or part nearest to the operator of the instrument and the term “distal” means the end or part furthest from the operator. Thus, the front end of the instrument that enters the body canal is the distal end.

The distal needle 8 comprises a proximal end 15, a distal end 17, and a lumen 19 extending from the proximal end 15 to the distal end 17. The distal end 17 of the distal needle 8 preferably comprises a cutting edge needle tip, which is suitable for puncturing skin and other soft tissues such as muscle tissue. In an alternative embodiment, the distal end 17 of the distal needle 8 may comprise a round point needle tip for use in connection with more delicate surgical operations. The intermediate tube 10 comprises a proximal end 22, a distal end 23, and a lumen 26 extending from the proximal end 22 to the distal end 23. The proximal tube 13 comprises a proximal end 29, a distal end 31, and a lumen 33 extending from the proximal end 29 to the distal end 31.

As presently embodied, the distal needle 8, the intermediate tube 10, and the proximal tube 13 all comprise surgical stainless steel, such as 304 grade surgical stainless steel or 316 grade surgical stainless steel. In accordance with one embodiment of the present invention, at least two needles (e.g., the distal needle 8 and the intermediate tube 10) are attached from larger to smaller diameter so as to create a transitional cone to facilitate the movement of viscous bulking material, such as material containing suspended beads or micro-spheres, through the transition-bore needle apparatus in a direction from the larger diameter tube to the smaller diameter needle. In the presently preferred embodiment, three needles (i.e., the distal needle 8, the intermediate tube 10 and the proximal tube 13) are attached, preferably using an adhesive 34, from larger to smaller diameters so as to create a transitional cone to facilitate the movement of viscous materials through the transition-bore needle apparatus in a direction from the larger diameter needle to the smaller diameter needles. Other modified embodiments may incorporate a greater number of needles.

The lumen of the transition-bore needle apparatus 7 comprises both a portion of the lumen 19 of the distal needle 8 and a portion of the lumen 26 of the intermediate tube 10, as can be seen in FIG. 1. A juncture thus exists within the lumen of the transition-bore needle apparatus 7, where the diameter thereof transitions from a diameter of the intermediate tube 10 to a diameter of the distal needle 8. At this juncture, the proximal end 15 of the distal needle 8 terminates within the lumen 26 of the intermediate tube 10.

In the illustrated embodiment, the distal needle 8 comprises an inner diameter of about 0.008 inches and an outer diameter of about 0.016 inches. The distal needle 8 fits into the intermediate tube 10, which in the illustrated embodiment comprises an inner diameter of about 0.020 inches and an outer diameter of about 0.028 inches. In the presently preferred embodiment, the distal needle 8 protrudes distally about 3 mm from the intermediate tube 13.

The intermediate tube 10 fits into the proximal tube 13, which as presently embodied comprises an inner diameter of about 0.50 inches, an outer diameter of about 0.032 inches, and a length of about 12 inches. The proximal tube 13 encloses the proximal end 22 of the intermediate tube 10. The proximal tube 13 preferably comprises three hypotubes, which may facilitate a tighter fit around the intermediate tube 10 and/or greater rigidity of the proximal tube 13.

A tissue stop 31 is preferably disposed about the distal needle 8 next to the distal end 23 of the intermediate tube 10. The tissue stop 31 preferably comprises a diameter, which is about the same as the diameter of the proximal tube 13. In alternative embodiments, other diameters may be constructed. The tissue stop 31 preferably comprises a circular perimeter, but may have oval or rectangular perimeters in alternative embodiments. The tissue stop 31 preferably comprises a polymeric material, which is more flexible than, for example, stainless steel. In modified embodiments, the tissue stop 31 may comprise surgical stainless steel.

An angle between a plane of the tissue stop 31 and a longitudinal axis of the transition-cone needle assembly 7 is preferably less than ninety degrees and, preferably, less than about seventy-five degrees and, more preferably, about sixty degrees as shown in FIG. 1. The orientation of the tissue stop 31 is preferably selected so that a planar surface of the tissue stop will align longitudinally with the axis of the particular lumen that is being treated. In other words, a planar surface of the tissue stop 31 should rest flat on the surface of the tissue that is to be treated with the distal needle 8. The tissue stop 31 will help to prevent the needle from penetrating deeper into the tissue than is required or desired. A surgeon performing an injection procedure using, for example, a cystoscope or the device disclosed in U.S. patent application Ser. No. 09/825,484, entitled URETHRA SURGICAL DEVICE, can view the tissue stop 31 for assistance in performing the injection at the proper angle and at the proper depth.

In a modified embodiment of the apparatus, the tissue stop 31 may be omitted so that only the difference in outer diameters between the distal needle 8 and the intermediate tube 10 effectively operate as a tissue stop. In yet another modified embodiment, the tissue stop can be secured about the intermediate tube 10, instead of being secured about the distal needle 8, so that a distal planar surface of the tissue stop is flush with the distal end 23 of the intermediate tube 15.

In another embodiment, a depth indicator can be disposed about the distal needle 8 next to the distal end 23 of the intermediate tube 10. A depth indicator can consist of a marked band or bands at a predetermined location away from the tip of the needle. The mark band can be applied to distal needle 8 by an easily visually detectable coating such as paint or a band of material secured to needle 20, or can be etched or otherwise manufactured into the needle. Other means and manners of making of a visually discernible marking are also possible. A depth indicator can be a guide or a gauge attached, such as slidably joined, to the distal needle 8.

According to another aspect of the present invention, the proximal end 22 of the intermediate tube 10 is beveled to improve a flow of viscous material through the lumen of the transition-bore needle apparatus 7. FIG. 1A is a cross-sectional view of a proximal end 22 with edges beveled at a 45 degree angle from the longitudinal axis of the distal needle 8. The beveling may be performed by filing an initially square outer edge to an angle less than ninety degrees and, preferably, less than sixty degrees, and more preferably, about forty-five degrees. After the proximal end 22 of the intermediate tube 10 is beveled, the intermediate tube 10 is then secured within the lumen 33 of the proximal tube 13, through the application of adhesive material between the outer surface of the intermediate tube 10 and inner surface of the proximal tube 13 at their contacting surfaces. In modified embodiments, the angle may be reduced to, for example, 30 degrees or even about 15 degrees from the longitudinal axis of the intermediate tube 10.

In accordance with another aspect of the present invention, the proximal end 22 of the intermediate tube 10 is chamfered to improve a flow of viscous material through the lumen of the transition-bore needle apparatus 7. FIG. 1B is a cross-sectional view of a proximal end 22 with edges chamfered at a 45 degree angle from the longitudinal axis of the distal needle 8. The chamfering may be performed by filing an initially square edge on the interior side of the tubing to an angle less than ninety degrees and, preferably, less than sixty degrees, and more preferably, about forty-five degrees. In modified embodiments, the angle may be reduced to, for example, 30 degrees, or even about 15 degrees from the longitudinal axis of the distal needle 8.

In yet another aspect of the present invention, the proximal end 22 of the intermediate tube 10 is both chamfered and beveled, in accordance with the structures discussed in the preceding paragraphs, to thereby improve a flow of viscous material through the lumen of the transition-bore needle apparatus 7. The proximal end 15 of the distal needle 8 is preferably beveled and/or chamfered, similarly to that described above in connection with the proximal end 22 of the intermediate tube 10. In modified embodiments, only the proximal end 15 of the distal needle 8 is beveled and/or chamfered, and the proximal end 22 of the intermediate tube 10 is neither beveled nor chamfered.

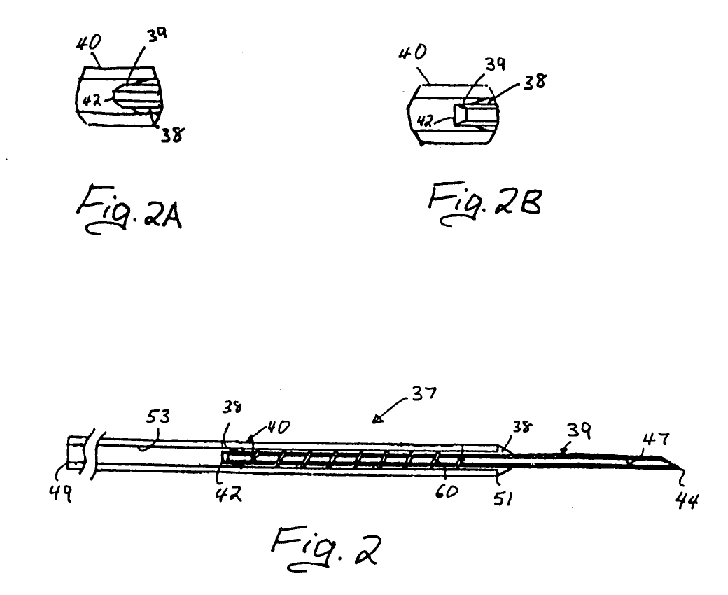

Turning now to FIG. 2, a transition-bore needle assembly 37 is illustrated for use in applications such as lower esophageal injections of bulking material. The transition-bore needle assembly 37 comprises a needle 39 connected to a flexible tube 40. The flexible tube 40 may comprise a polymeric material, such as polyethylene terephthalate (PET). The needle 39 comprises a proximal end 42, a distal end 44, and a lumen 47 extending between the proximal end 42 and the distal end 44. The distal end 44 of the needle 44 preferably comprises a cutting edge needle tip, which is suitable for puncturing skin and other soft tissues such as muscle tissue. As presently embodied, the cutting edge needle tip is formed at a 20 degree angle from a longitudinal axis of the needle 39. In an alternative embodiment, the distal end 44 of the needle 39 may comprise a round point needle tip. The flexible tube 40 similarly has a proximal end 49, a distal end 51, and a lumen 53 extending from the proximal end 49 to the distal end 51.

As presently embodied, the needle 39 comprises surgical stainless steel, such as 304 grade surgical stainless steel or 316 grade surgical stainless steel. The proximal end 42 of the needle 39 is inserted into and attached to the distal end 51 of the flexible tube 40, so as to create a transitional cone to facilitate the movement of viscous bulking material, such as material containing suspended beads or micro-spheres, through the transition-bore needle apparatus 37 in a direction from the flexible tube 40 to the needle 39. The lumen of the transition-bore needle apparatus 37 comprises both a portion of the lumen 47 of the needle 39 and a portion of the lumen 53 of the flexible tube 40, as can be seen from FIG. 2. A juncture thus exists within the lumen of the transition-bore needle apparatus 37, where the diameter thereof transitions from a diameter of the flexible tube 40 to a diameter of the needle 39. At this juncture, the proximal end 42 of the needle 39 terminates within the lumen 53 of the flexible tube 40.

In the illustrated embodiment, the needle 39 has an inner diameter of about 0.012 inches and an outer diameter of about 0.020 inches, and the flexible tube 40 has an inner diameter of 0.032 inches and an outer diameter of 0.056 inches. A wire 60 is wrapped around the needle 39 and glued into place. In the gluing process, the glue 38 is preferably allowed to dry and then heat cured. Another glue is then applied to the surfaces of the resulting wire 60 and needle 39. The glue may comprise, for example, a lock-tight glue or a superglue. The needle 39 is then gripped and held with, for example, a chuck, and screwed into the flexible tube 40, which may comprise a polymeric tube having, for example, a smooth inner surface. Some glue will remain on the distal end 51 of the flexible tube 40 to form a seal, after the needle 39 and wire 60 are screwed in. The wire 60 around the needle 39 preferably deforms the smooth inner surface of the flexible tube 40 for a frictional fit.

According to another aspect of the present invention, the proximal end 42 of the needle 39 is beveled to improve a flow of viscous material through the lumen of the transition-bore needle apparatus 37. FIG. 2A is a cross-sectional view of a proximal end 42 with edges chamfered at about a 30 degree angle from the longitudinal axis of the distal needle 39. The beveling may be performed by filing an initially square edge to an angle less than ninety degrees and, preferably, less than sixty degrees, and more preferably, about forty-five degrees. After the proximal end 42 of the needle 39 is beveled and/or chamfered, the needle 39 is secured within the lumen 53 of the flexible tube 40. In modified embodiments, the angle may be reduced to, for example, 30 degrees or about 15 degrees.

In accordance with another aspect of the present invention, the proximal end 42 of the needle 39 is chamfered to improve a flow of viscous material through the lumen of the transition-bore needle apparatus 37. FIG. 2B is a cross-sectional view of a proximal end 42 with edges chamfered at about a 30 degree angle from the longitudinal axis of the distal needle 39. The chamfering may be performed by filing an initially square edge to an angle less than ninety degrees and, preferably, less than sixty degrees, and more preferably, about forty-five degrees. In modified embodiments, the angle may be reduced to, for example, 30 degrees, or even about 15 degrees. According to yet another aspect of the present invention, the proximal end 42 of the needle 39 is both chamfered and beveled, in accordance with the structures discussed in this preceding paragraph, to thereby improve a flow of viscous material through the lumen of the transition-bore needle apparatus 37.

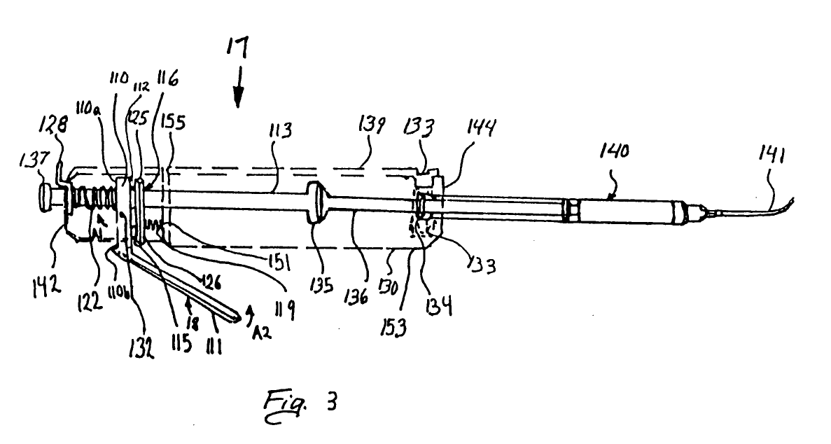

FIG. 3 illustrates an injection facilitation apparatus 17 for use with the injection apparatus of the present invention. Injection facilitation apparatus 17 may be used with the transition-bore needle apparatus disclosed hereinabove. The housing 130 preferably comprises a molded polymeric material, generally in the shape of a hollow cylinder. In modified embodiments, other materials and shapes may be used. In the illustrated embodiment, the housing 130 comprises a proximal end 142, a distal end 144, a first side 139 and a second side 153.

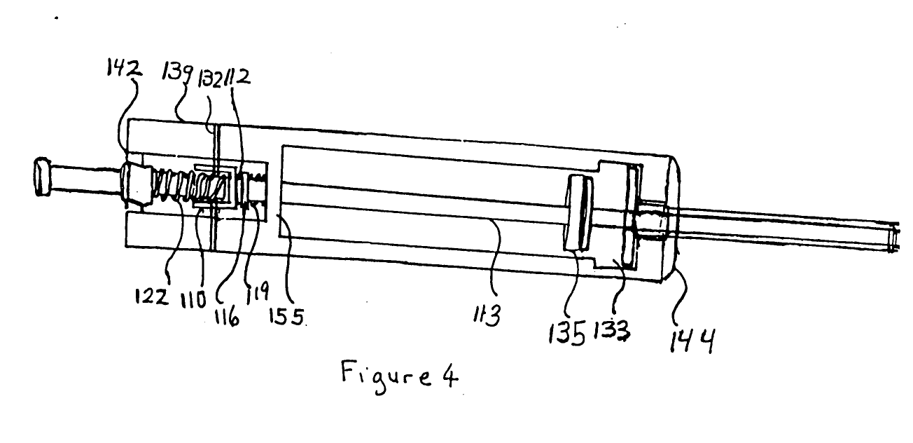

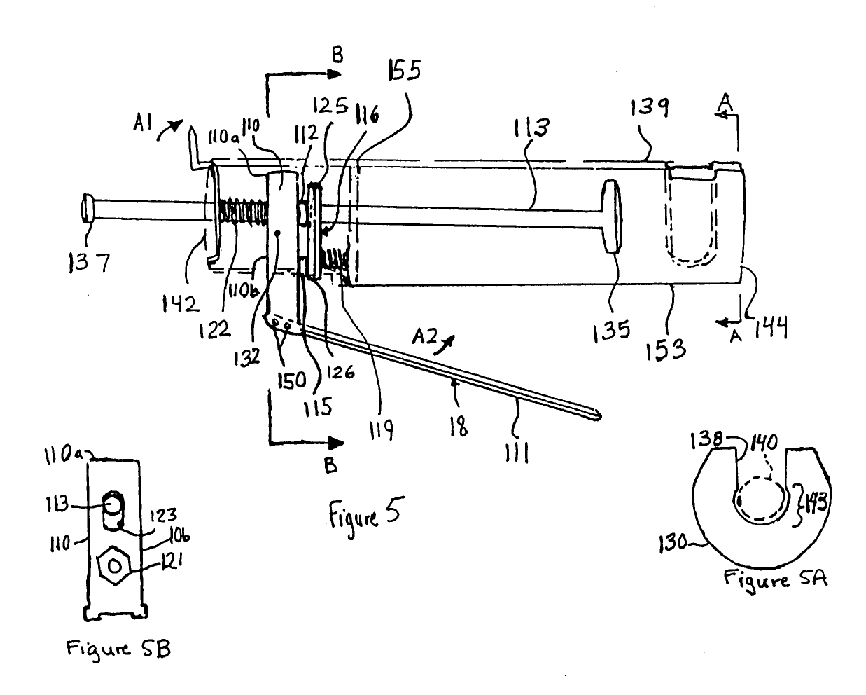

FIGS. 4 and 5 are additional views of the injection facilitation apparatus 17. A portion of the housing 130 is preferably removed on the first side to accommodate a syringe 140 therein. In modified embodiments, other means, such as a hinged door, may be incorporated for accommodating the syringe 140 into the housing 130. The housing 130 comprises a slot 133, extending in a direction from the first side to the second side into the housing and being configured to accommodate and hold a finger rest 134 of the syringe 140 within the housing. An end view of the housing 130, taken in the direction of the arrow A-A of FIG. 5, is shown in FIG. 5A. A slot 138 is disposed at the distal end 144 of the housing 130 for accommodating the syringe 140 therein. The slot 138 extends from the first side 139 of the housing in a direction toward the second side 153, and terminates with a circular or rounded portion 143 for accommodating and holding the syringe 140.

A movable rod 113 extends within the housing 130 from the proximal end 142 to the distal end 144 (in accordance with a fully extended position of the rod 113). When the rod 113 pushes distally against the syringe 140, the syringe 140 will tend to move in the distal direction. The finger rest 134 of the syringe 140, however, will press distally against the slot 133, to thereby prevent distal movement of the syringe 140. Thus, only the plunger 136 of the syringe 140 will move distally under the distal force of the rod 113. Distal movement of the plunger 136, relative to the rest of the syringe 140, will result in the plunger 136 expelling a portion of the syringe contents, which may comprise a viscous bulking agent, from the syringe 140 and into the elongate catheter 141.

The elongate catheter 141 preferably comprises a length sufficient to extend through the relevant body passage and to the site of operation. For example, the elongate catheter 141 may be constructed to have a length sufficient to extend through a cystoscope for operation on the urinary sphincter near the bladder. As another example, the elongate catheter 141 may be configured to have a length (e.g., about one meter) sufficient to extend through a gastroscope for operation on the lower esophageal sphincter near the stomach. The elongate catheter 141 may comprise or be connected to the flexible tube 13 of FIG. 1 or the proximal tube 40 of FIG. 2.

The force required for delivery of a viscous fluid through the elongate catheter 141 and to the target site of injection will be proportionate to the length and cross-sectional area of the elongate catheter 141. This force required to deliver the viscous material through the lumen of the elongate catheter will thus increase as the length of the elongate catheter increases, and further will increase as the cross-sectional area of the elongate catheter is decreased.

The injection facilitation apparatus 17 of the present invention facilitates the injection of viscous filler materials, and provides for increased accuracy in the amounts of such dispensed materials. Filler materials can be those agents that can temporarily or permanently fill a void. Filler materials, such as soft tissue fillers, have a variety of applications for tissue augmentation and tissue bulking. Certain filler materials can also be used to coat a lumen surface to prevent collapse of the lumen or repair tears or other like damage. Filler materials include those with adhesive properties and those that can be mixed with adhesive agents. Examples of filler materials are ArteColl® and ArteFill®, which are trade names for tissue bulking or filling agents, such as collagen-suspended microspheres, which can be formed of polymers such as polymethyl methacrylate (PMMA). ArteFill® is approximately 20% by weight PMMA and approximately 80% by weight a composition of 3.5% purified bovine collagen, 2.7% phosphate buffer, 0.9% sodium chloride, 0.3% lidocaine hydrochloride, and 92.6% water for injection. Examples of microsphere-based filler materials are disclosed in U.S. Pat. No. 5,344,452, which is incorporated herein by reference. Other tissue fillers include, but are not limited to, collagen; polylactic acid, polymethacrylate, polypropylene, polytetrafluoroethylene (PTFE); hollow cylinder pellets such as disclosed in U.S. Patent Publication No. 2004/210230, entitled “Materials and Methods for Soft Tissue Augmentation”; polysaccharide-based gel such as disclosed in U.S. Patent Publication No. 2004/0047892, entitled “Filler Composition for Soft Tissue Augmentation and Reconstructive Surgery”; polyhydroxyalkanoate materials such as disclosed in U.S. Pat. Nos. 6,585,994 and 6,555,123, entitled “Polyhydroxyalkanoate Compositions for Soft Tissue Repair, and Viscosupplementation”; hyaluronic acid including such as disclosed in U.S. Pat. No. 5,827,937; repetitive protein polymers such as disclosed in U.S. Patent Publication No. 2003/0176355, entitled “Synthetic Proteins for In Vivo Drug Delivery and Tissue Augmentation”; a three-part injectable polymer such as disclosed in U.S. Pat. No. 5,785,642; a two-part injectable polymer such as disclosed in U.S. Pat. No. 6,312,725; keratin such as disclosed in U.S. Pat. No. 5,712,252; ceramic microsphere compositions such as disclosed in U.S. Pat. Nos. 5,922,025 and 6,432,437, and 6,537,574, entitled “Soft Tissue Augmentation Material”; biocompatible tissue-reactive prepolymer such as disclosed in U.S. Pat. No. 6,702,731, entitled “Situ Bulking Device”; cross-linked blood plasma proteins such as disclosed in U.S. Pat. No. 7,015,198, entitled “Materials for Soft Tissue Augmentation and Methods of Making and Using Same”; radiation cross-linked hydrogels such as disclosed in U.S. Pat. No. 6,537,569, entitled “Radiation Cross-Linked Hydrogels”; bioelastomers such as disclosed in U.S. Pat. Nos. 6,533,819 and 6,699,294, entitled “Injectable Implants for Tissue Augmentation and Restoration”; cross-linked water-swellable polymer particles such as disclosed in U.S. Pat. Nos. 6,214,331 and 6,544,503, entitled “Process for the Preparation of Aqueous Dispersions of Particles of Water-Soluble Polymers and Particles Obtained”; and compositions including a pseudoplastic polymer carrier such as disclosed in U.S. Pat. No. 5,633,001, entitled “Composition and a Method for Tissue Augmentation”. Filler materials can be cross-linked or not cross-linked. or made of a synthetic and/or polymeric material, such as, for example, polylactic acid, organic compounds, inorganic compounds, ceramic materials, polymethacrylate, PMMA, polypropylene, polytetrafluoroethylene (PTFE), and combinations thereof. Filler materials can be used as bulking agents, wherein the filler mechanically fortifies a body tissue, for example, an anatomic structure such as a sphincter muscle.

A filler material can have a short-term effectiveness of up to about 6 months, a medium-term effectiveness of up to about 3 years, and/or a long-term effectiveness of about 3 years or more, depending upon the nature of the filler injected. The filler material can be microparticles. The microparticles can be made at least in part of biological materials, such as, for example, but not limited to, one or more of any type of collagen, hyaluronic acid (e.g., animal derived, human derived and/or tissue/cell culture derived), cells, tissues, organisms, genetically altered or not (e.g., purified cytoskeleton of unicellular and/or multicellular algae and/or other organisms), whether cross-linked or not cross-linked, or made of a synthetic and/or polymeric material, such as, for example, polylactic acid, organic compounds, inorganic compounds, ceramic materials, polymethacrylate, PMMA, polypropylene, polytetrafluoroethylene (PTFE), and combinations thereof.

Filler compositions as disclosed supra can further include compositions with materials that aid in growth or suppress growth of the injected or surrounding tissues. For example such embodiments can include compositions comprising autologous body components and fluids as disclosed in co-owned U.S. patent Ser. No. 11/210,273, entitled “Methods of Administering Microparticles Combined With Autologous Body Components”, herein incorporated by reference. Alternatively, one can prepare a composition comprising cells and a filler material. Cells can be autogeneic, isogeneic, allogeneic or xenogeneic. Cells can be genetically engineered. The compositions can contain different cell types, which may be chosen to act synergistically, for example, in the formation of tissue. Examples of types of cells include muscle cells, nerve cells, epithelial cells, connective tissue cells, and organ cells. Specific examples of cells include fibroblast cells, smooth muscle cells, striated muscle cells, heart muscle cells, nerve cells, epithelial cells, endothelial cells, bone cells, bone progenitor cells, bone marrow cells, blood cells, brain cells, kidney cells, liver cells, lung cells, pancreatic cells, spleen cells, breast cells, foreskin cells, ovary cells, testes cells and prostate cells. The types of cells include stem cells, which can be fetal stem cells or adult stem cells and can be totipotent, multipotent, or pluripotent. Other mammalian cells are useful in the practice of the invention and are not excluded from consideration here. Alternatively, the filler material compositions can include non-mammalian eukaryotic cells, prokaryotic cells or viruses.

Filler compositions can include physiologically buffered salt solutions, water, glycerol and the like, and may be supplemented with, for example, serum, growth factors, hormones, sugars, amino acids, vitamins, metalloproteins, lipoproteins, and the like.

Growth factors include, but are not limited to, transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Members of the TGF supergene family include the beta transforming growth factors (for example, TGF-.beta.1, TGF-.beta.2, TGF-.beta.3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB).

Growth factors can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

An exemplary embodiment of the invention comprises an injection facilitation apparatus for use in conjunction with a needle tip stainless steel elongate catheter that can be introduced through a subject's urethra in the treatment of urinary incontinence. As another example, a treatment for gastro-esophageal reflux disease may be fashioned to increase the strength or length of the lower esophageal sphincter (LES) via the deposition of a viscous bulking material into surrounding tissues of the lower esophageal sphincter. An injection facilitation apparatus of the present invention is suitable for such use in conjunction with a needle tip, flexible, polymeric elongate catheter. The viscous suspension can be injected via a syringe and needle directly into the specific areas where the viscous agent is desired. Principal uses of the present invention are to accurately and conveniently dispense the viscous material to thereby alter the operational architecture of the subject's sphincter. Thus, the bio-mechanical characteristics of the sphincter are altered to alleviate the disorder.

As shown in FIG. 3, a fastener 128 comprises an aperture for accommodating the rod 113 therethrough, and is biased proximally against an inner surface of the proximal end 142 of the housing 130. In the illustrated embodiment, the fastener 28 comprises surgical stainless steel. A handle spring 122 is disposed about the rod 113 between an internal end 110 of a pivot arm 18 and the fastener 128. In addition to comprising an internal end 110, the pivot arm 18 further comprises a handle end 111. The handle spring 122 provides a proximal biasing force against the fastener 128 and a distal biasing force against the internal end 110 of the pivot arm 8. The handle spring 122 may comprise, for example, surgical stainless steel.

It can be seen from the figure that the handle spring 122 biases an internal first end 110a of the pivot arm 18 distally, and the driving spring 119 biases an internal second end 110b of the pivot arm 18 proximally. The combination of the handle spring 122 and the driving spring 119 tend to rotate the pivot arm 18 about the pivot pin 132 in the clockwise direction shown by the arrow A1. An inward thrust by the hand of a user on the handle end 111 of the pivot arm 18 causes the pivot arm 18 to rotate about the pivot pin 132 in the direction of the arrow A2. As the pivot arm 18 pivots about the pivot pin 132, the internal first end 110a of the pivot arm 18 moves generally in a proximal direction and the internal second end 110b of the pivot arm 18 moves generally in a distal direction. When the internal second end 110b of the pivot arm 18 moves distally in response to the handle end 111 moving in the direction of the arrow A2, the screw head 115, which is secured to the internal second end 110b, applies a distal force against the biased end 126 of the of the driving arm 116.

In the illustrated embodiment, the handle end 111 is attached to the internal end 110 via two small identical screws 150 (FIG. 5), and the internal end 110 rests on the handle spring 122. As can be seen best in FIG. 3, a pivot pin 132, preferably comprising a surgical stainless steel bar, passes through both the housing 130 and the internal end 110 of the pivot arm 18. The pivot arm 18 thus pivots about the pivot pin 132 in both clockwise and counter-clockwise directions, as shown in FIG. 3 by the arrows A1 and A2, respectively.

A ring 112, which preferably comprises surgical stainless steel, is disposed around the rod 113 between the internal end 110 and a driving arm 116. The ring 112 preferably comprises a loosely fitted and movable stainless steel washer. The driving arm 116, preferably comprising stainless steel, rests on top of the ring 112. In between the driving arm 116 and the internal end 110 also rests a screw head 115, positioned on a distal side of the internal end 110. The screw head 115 is secured onto the internal end 110 of the pivot arm 18 with a nut 121, as can be seen from FIG. 5b. Distally of the driving arm 116 rests the driving spring 119, which is held in place by an alignment protrusion 151 that preferably comprises a knob (not shown) formed on a housing wall 155 of the housing 130

The rod 113 extends through the fastener 128, the handle spring 122, the pivot arm 18, the ring 112, and driving arm 116. The internal end 110 of the pivot arm 18 and the driving arm 116, in combination with the fastener 128, work together to provide slidable alignment to the rod 113. The rod 113 comprises a proximal rod disk 137 connected at a proximal end of the rod 113 and a distal rod disk 135 connected at a distal end of the rod 113. As presently embodied, the rod 113 is removably attached to at least one of the proximal rod disk 137 and the distal rod disk 135. In the illustrated embodiment, the rod 113 is permanently secured to or integrally formed with the proximal rod disk 137 and is removably connected (e.g., threaded) to the distal rod disk 135.

The injection facilitation apparatus 17 contains a pivot mechanism that forces the rod 113 distally within the housing 130 toward the slot 133, where the syringe 140 is held and positioned, as illustrated in FIG. 3. A clear depiction of the slot 133 can be seen in FIG. 4. When the syringe 140 is positioned within and held by the housing 130, a base of the syringe, plunger 136, rests flat against the distal rod disc 135.

FIGS. 3-5 depict the injection facilitation apparatus 17 at rest, wherein no external force is applied to the handle end 111 of the pivot arm 18. In the relaxed or resting position of FIGS. 3-5, there is no gap between the driving arm 116 and the ring 112, which rests on the internal end 110 of the pivot arm 18. It can be seen from the figure that without an external force applied to the handle end 111 of the pivot arm 18, the internal end 110 of the pivot arm 18 rests between the handle spring 122 and a combination of the ring 112 and the driving arm 116. The handle spring 122 biases an internal first end 110a of the pivot arm 18 distally, and the driving spring 119 biases an internal second end 110b of the pivot arm 18 proximally.

The combination of the handle spring 122 and the driving spring 119 tend to rotate the pivot arm 18 about the pivot pin 32 in the clockwise direction shown by the arrow A1. However, in accordance with an aspect of the present invention, a rotation-limiting structure prevents the pivot arm 18 from rotating clockwise past the orientation shown in FIG. 3 and FIG. 5. For this reason, the orientation of the pivot arm 18 shown in, for example, FIG. 3 is referred to as being in the relaxed position. Application of a force by the hand of an operator to pivot the handle end 111 of the pivot arm 18 in the counter-clockwise direction shown by arrow A2 will move the assembly out of the relaxed position and move the rod 113 distally.

Regarding the rotation-limiting structure and with reference to FIG. 5, the handle spring 122 applies a distal force onto the internal first end 110a, resulting in the generation of a rotational force or moment arm on the internal end 110 which would cause the internal end 110 to rotate about the pivot pin 132 in the direction of the arrow A1. If the pivot pin 132 were to rotate in the direction of the arrow A1, the internal first end 110a would pivot distally about the pivot pin 132 and the internal second end 110b would pivot proximally about the pivot pin 132. When the internal end 110 of the pivot arm 18 is in the resting position as shown in FIGS. 3-5, however, an aperture 123, which is disposed on the internal end 110 of the pivot arm 18 for accommodating the rod 113 therethrough, prevents rotation in the direction of the arrow A1. An end view of the internal end 110, taken in the direction of the arrow B-B of FIG. 5, is shown in FIG. 5B. The aperture 123, which serves as a rotation-limiting structure, is shaped to (1) allow pivoting of the internal end 110 from the position shown in FIGS. 3-5 in the direction of the arrow A2, and (2) prevent pivoting of the internal end 110 from the position shown in FIGS. 3-5 in the direction of the arrow A1.

More particularly, the aperture 123 comprises an elongate shape having a width that is about the same dimension as a diameter of the rod 113 passing through the aperture 123, and having a length that is appreciably greater than the diameter of the rod 113 passing through the aperture 123. For example, the length should be at least 1.25 times the diameter of the rod 113 passing through the aperture 123 and, preferably, should be at least about 1.5 times, and more preferably, should be about 2 times the diameter of the rod 113 passing through the aperture 123.

Now, regarding the orientation of the aperture 123 relative to the rod 113 passing therethrough, in the position shown in FIG. 5 the portion of the aperture 123 closest to the internal first end 110a of the pivot arm 18 contacts the rod 113 to prevent further movement of the pivot arm 18 in the direction of the arrow A1. However, in the same position shown in FIG. 5, the portion of the aperture 123 closest to the internal second end 110b of the pivot arm 18 does not contact and is spaced from the rod 113 to facilitate movement of the pivot arm 18 in the direction of the arrow A2. Thus, the engagement of the portion (e.g., edge) of the aperture 123 closest to the internal first end 110a with the rod 113 serves to limit rotational movement of the pivot arm 18 in the direction of the arrow A1. In modified embodiments, other constructions may be used to limit rotational movement of the pivot arm 18 in the direction of the arrow A1, such as a protrusion on the second side 153 of the housing 130 to contact the internal second end 110b and prevent that end from moving proximally from the position shown in FIG. 5.

The effect of a pivoting force applied by the hand of a user on the pivot arm 18 of the injection facilitation apparatus 17 can be seen through a comparison of FIGS. 5-7. An inward thrust by the hand of a user on the handle end 111 of the pivot arm 18 causes the pivot arm 18 to rotate about the pivot pin 132 in the direction of the arrow A2. As the pivot arm 18 pivots about the pivot pin 132, the internal first end 110a of the pivot arm 18 moves generally in a proximal direction and the internal second end 110b of the pivot arm 18 moves generally in a distal direction. When the internal second end 110b of the pivot arm 18 moves distally in response to the handle end 111 moving in the direction of the arrow A2, the screw head 115, which is secured to the internal second end 110b, applies a distal force against the biased end 126 of the of the driving arm 116.

As the screw head 115 moves generally distally, while rotating about the pivot pin 132, the screw head 115 slides against the driving arm 116 in a direction toward the rod 113. The component of movement of the screw head 115 in the distal direction, as it slides on the driving arm 116, moves the biased end 126 of the driving arm 116 distally against the proximal bias of the driving spring 119. The changes in position of the pivot arm 18 and the driving arm 116 can be seen through the progression of FIGS. 5-7.

The initial distal movement of the screw head 115 is applied to the biased end 126 of the driving arm 116, which generates a moment arm. The moment arm tends to cause the driving arm 116 to pivot generally in the direction of the arrow A2 and generally about a pivot point defined by the interaction of the rod 113 and the driving arm 116.

A rod-clamping end 125 of the driving arm 116 comprises an aperture for accommodating the rod 113 therethrough. The cross-sectional area of the aperture is shaped to be slightly larger than the cross-sectional area of the rod 113, so that the rod 113 can freely move through the aperture when the driving arm 116 is oriented at a predetermined orientation (corresponding, for example, to an orientation when the pivot arm 18 is in the relaxed position). As presently embodied, the rod 113 can freely move through the aperture of the driving arm 113, with little or reduced friction relative to other orientations, when the driving arm 113 is substantially perpendicular to an axis of the rod 113. In modified embodiments, other angular positions may be implemented.

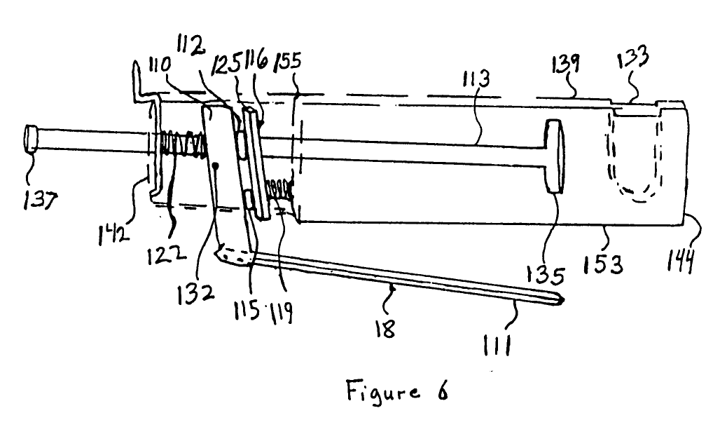

As the driving arm 116 moves under the influence of the screw head 115 from its perpendicular orientation in an approximate direction of the arrow A2, biased end 126 of the driving arm 116 will move slightly distally so that the orientation of the driving arm 116 changes and so that the rod 113 can no longer freely slide (or slide with reduced friction) through the aperture of the driving arm 116. Thus, the clamping end 125 of the driving arm 116 will be somewhat locked onto the rod 113 at the angle of contact (which as presently embodied is an angle of about one to a about five degrees from 90 degrees). At this position, shown in FIG. 6, there will be a small gap between internal end 110 and driving arm 116.

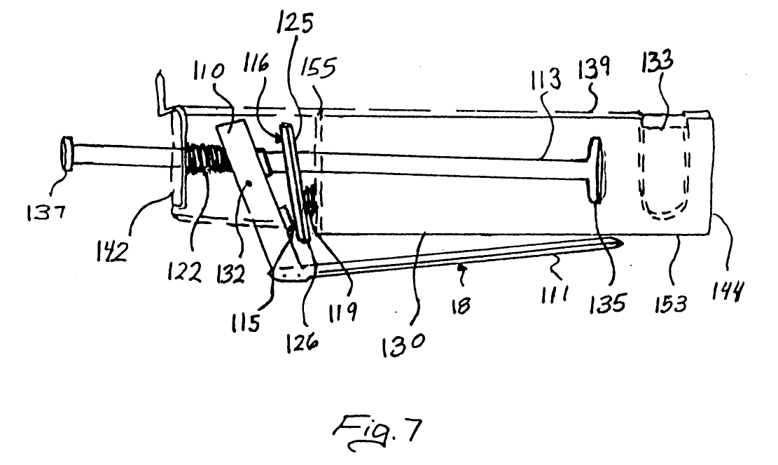

Further movement of the handle end 111 in the direction of the arrow A2 is depicted in FIG. 7. During this extended push on pivot arm 18 the distance between internal end 110 and driving arm 116 will increase to its maximum separation. The screw head 115 continues to move the driving arm 116 distally. Since the driving arm 116 has already rotated slightly to clamp the rod 113, the driving arm 116 will not rotate further as the screw head 115 pushes further and further distally against the driving arm 116. Thus, continued movement of the screw head 115 against the driving arm 116 moves the driving arm 116, which in turn moves the clamped rod 113 forward.

As the driving arm 116 moves forward, the driving spring 119 is compressed against the housing wall 155, and the handle spring 122 is compressed proximally against the fastener 128 by proximal movement of the internal first end 110a. In the illustrated embodiment, movement of the pivot arm 18 in the direction of the arrow A2 will cease when the handle end 111 contacts the surface of the housing 130.

In modified embodiments of the invention the length of the internal end 110 can be altered. Varying the length of the internal end 110 can vary the amount of material expelled from the catheter-syringe within the injection facilitation apparatus. For example, the internal end 110 can be lengthened such that the new added length protrudes out of the housing on the second side 153, which will cause a proportional increase in the range of movement of the handle end 111. In accordance with another modification, the angle formed between the handle end 111 and the internal end 110 can be increased so that the handle end 111 can be moved further in the direction of the arrow A2 for a corresponding greater movement of the rod 113. In accordance with one aspect of the present invention, the injection facilitation apparatus is engineered so that a full compression of the handle end 111 will eject an exact amount of filler material, such as a specific volume amount to generate one mucosa bulge near a urinary sphincter.

As the angle between the handle end 111 and the internal end 110 increases, the amount of distance created between the internal end 110 and the driving arm 116 at maximum compression of the handle end 111 is also intensified. This translates into a greater range of push movement on the rod 113, and thus a greater amount of viscous material being expelled from the syringe as the plunger 136 receives the additional push from the rod 113.

In other words, the screw head 115 moves generally distally, to thereby move the biased end 126 of the driving arm 116 distally against the proximal bias of the driving spring 119. The initial distal movement of the screw head 115 is applied to the biased end 126 of the driving arm 116, which generates a moment arm. The moment arm tends to cause the driving arm 116 to pivot generally in the direction of the arrow A2 and generally about a pivot point defined by the interaction of the rod 113 and the driving arm 116. As the driving arm 116 moves under the influence of the screw head 115 from its perpendicular orientation in an approximate direction of the arrow A2. The biased end 126 of the driving arm 116 will move slightly distally so that the orientation of the driving arm 116 changes and so that the rod 113 can no longer freely slide (or slide with reduced friction) through the aperture of the driving arm 116. Thus, the clamping end 125 of the driving arm 116 will be somewhat locked onto the rod 113 at the angle of contact (which as presently embodied is an angle of about one to a about five degrees from 90 degrees). Continued movement of the screw head 115 against the driving arm 116 moves the driving arm 116, which in turn moves the clamped rod 113 forward.

Although the injection facilitation apparatus 17 is designed to facilitate accurate dispensing of viscous materials from a syringe, such as filler materials, the injection facilitation apparatus 17 further may be used to accurately dispense other materials and fluids as well. In the illustrated embodiment, the filler material comprises collagen and/or micro-spheres, such as disclosed in U.S. Pat. No. 5,344,452, the contents of which are expressly incorporated herein by reference, or, for example, any other type of injectable bulking agent.

Another alternative embodiment would comprise a longer housing 120, with a correspondingly longer rod 113, such that the length of the rod 114 would extend proximally an additional distance equal to about a length of the handle end 111. The handle end 111 would then be attached via screws 150 to the internal end 110 in an inverted position, so that the handle end 111 extends proximally instead of distally. The handle can be formed of another shape to accommodate the different direction of action, and the mechanism of action and other components would remain substantially the same.

The injection facilitation apparatus 17 can increase the precision of dispensing fluids from the syringe 140, as it can be calibrated to permit a specific concentration of material to be dispensed from the syringe corresponding to a certain range of movement of the handle end 111. This is especially important due to the high level of viscosity of the material being passed through the syringe, the distance of the elongate catheter 141, and the general need for surgical precision when injecting bulking agents. Further, the injection facilitation apparatus 17 can facilitate effective dispensation by reducing the amount of strength or effort required to secrete the viscous material out the syringe.

The treatment for gastro-esophageal reflux disease may be fashioned to increase the strength or the length of the lower esophageal sphincter (LES) by depositing a viscous material around the lower esophageal sphincter. The suspension can be injected via a syringe and needle directly into the specific areas where the viscous agent is desired. A principal use of the exemplary embodiment is to accurately dispense the viscous material to thereby alter the physiological architecture of the subject's sphincter and adjacent tissues. Thus the bio-mechanical characteristics of the and sphincter surrounding tissues are altered to alleviate urinary incontinence and gastro-esophageal reflux.

The transition-bore needle apparatus 17 of the present invention facilitates the injection of the viscous filler material, by optimizing a flow of the viscous material at the junctions of the needles used for intraluminal injections. The transition-bore needle apparatus 17 may be used in conjunction with surgical instruments, such as endoscopes, cystoscopes, and gastroscopes, and the like, to aid in intraluminal injections of materials into or adjacent to body tissues within body lumens. A body lumen is an inner open space or cavity of a tubular organ. A body lumen can be a blood vessel, a duct, a bronchus, a lung, a sinus, an esophagus, a stomach, a duodenum, a small intestine, a large intestine, a colon, a rectum, a ureter, a urethra, a vagina, a fallopian tube, and the like. For example, when the body lumen comprises an esophagus, a gastroscope is inserted through the esophagus into a vicinity of the lower esophageal sphincter, and a long needle is used to inject a filler material into and adjacent to the lower esophageal sphincter tissues for the treatment of acid reflux. When the body lumen comprises a female urethra, a cystoscope is inserted through the urethra to the urinary sphincter adjacent to the bladder neck, and a long needle is used to inject a filler material into and adjacent to the urinary sphincter tissues for the treatment of stress urinary incontinence. The filler material may also be injected, for example, along a greater length of the urethra.

The methods and devices of the invention can be used on a subject possessing the relevant anatomic structure. The subject can be an animal. The animal can be a mammal. The mammal can be a human.

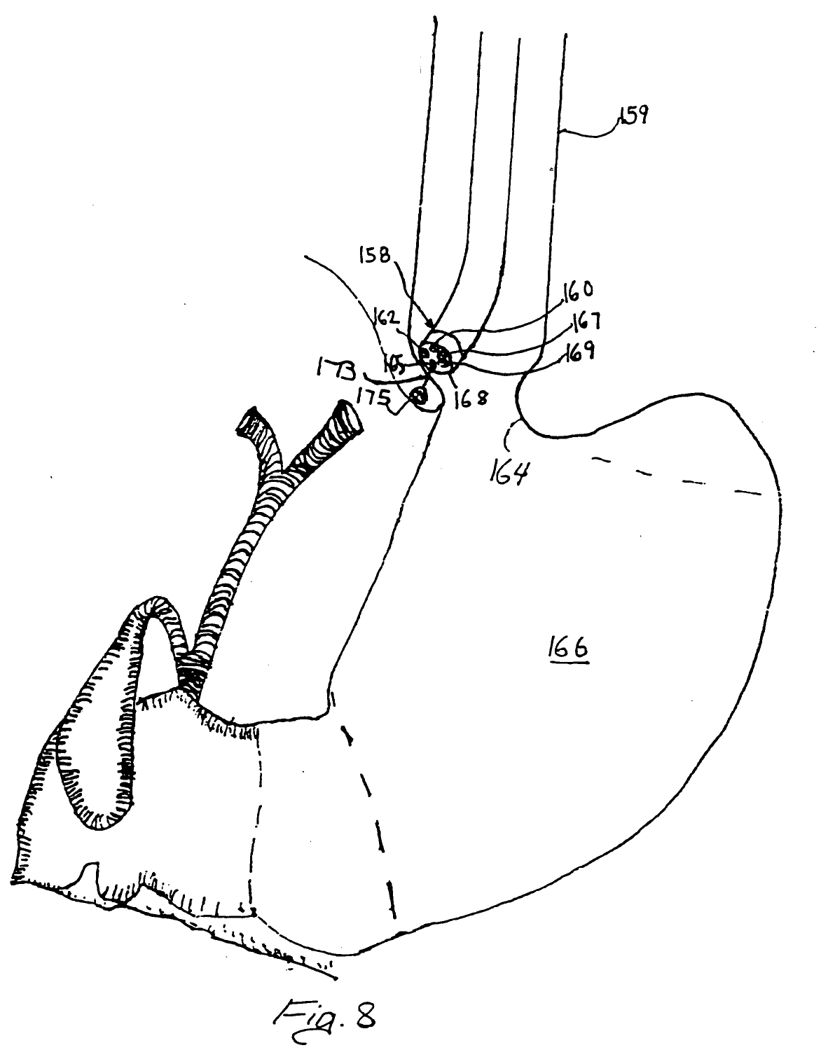

FIG. 8 illustrates a gastroscope 158 inserted through an esophagus 159 of a subject. The gastroscope 158 is positioned near the subject's lower esophageal sphincter 164 just above the body of the stomach 166. The injection facilitation apparatus 17 of the present invention is used in conjunction with a syringe and the gastroscope 158 of FIG. 8.

The gastroscope 158 in the illustrated embodiment is constructed to be flexible and to be capable of bending, for example, one hundred eighty degrees. Although other scopes and surgical devices suitable for insertion and manipulation within body passages may be used in accordance with the present invention, the presently illustrated surgical device comprises a gastroscope 158 having a flexible, cylinder body with a distal end 168 for facilitating surgical procedures within a body passage. In the illustrated embodiment, the gastroscope 158 comprises an Olympus GIF-K Gastroscope.

The distal end 168 in the illustrated embodiment comprises five openings, but as few as two openings may be incorporated in modified embodiments. An objective lens 160 is enclosed in a first one of the openings to provide a visual pathway through the lumen and of the surgical site of interest. The gastroscope 158 further comprises another opening for providing a suction and/or working channel 165. Also provided at the distal end 168 is a channel for accommodating a light guide 162, which carries light to the distal end 168 for facilitating viewing of the treatment area through the visual passageway. The light guide 162 preferably comprises a fiber optic light guide. Alternatively a LED, or other bulb, or other light source may be incorporated. A water-feeding nozzle 169, which directs pressurized water across the objective lens 160 to clear debris and an air-feeding nozzle 167 are also housed within two respective channels of the gastroscope 158. The air-feeding nozzle 167 can be used to direct pressurized air across the objective lens 160 to remove moisture and to provide, in accordance with one application, distension of the cavity being examined.

An elongated catheter, such as that shown at 141 in FIG. 3, is inserted through the working channel 165 for dispensing a somewhat viscous material into the surgical site, which in FIG. 8 comprises a vicinity of the lower esophageal sphincter 164. In the presently preferred embodiment, the elongate catheter 141 may have a length of, for example, about 1 meter to allow the elongate catheter to extend through the esophagus and to the lower esophageal sphincter 164.

The user presses the handle end 111 to thereby move the rod 113 distally against the syringe plunger 136. Distal movement of the plunger 136 forces viscous material within the syringe 140 distally out of the syringe 140 and through the elongate catheter 141. The elongate catheter 141 in the illustrated embodiment comprises a flexible material, such as a polymeric material, to facilitate maneuverability of the gastroscope 158. When the elongate catheter 141 is used in connection with a urethra procedure, such as the injection of bulking or filling material into a vicinity of a urinary sphincter, the elongate catheter 141 preferably comprises a surgical stainless steel. Injection procedures and apparatus, which utilize an elongate catheter and an accompanying syringe for treating, for example, urinary incontinence, and which are suitable for use with the injection facilitation apparatus 17 for urethral applications, are described in co-pending U.S. application Ser. No. 09/825,484, entitled URETHRA SURGICAL DEVICE, filed Apr. 2, 2001, the contents of which are expressly incorporated herein by reference.

A needle 173 is disposed at a distal end of the elongate catheter 141 for transferring viscous or other material from the elongate catheter 141 into tissue. The needle 173 penetrates into the tissue near the lower esophageal sphincter to inject a bulge 175 of bulking agent, as shown in FIG. 8. Additional bulking agent injections are formed around the lower esophageal sphincter 164 to thereby bulk up the tissue in the vicinity of the lower esophageal sphincter.

The needle preferably penetrates through the mucosa but not through the muscle layers of the lower esophageal sphincter 164, to thereby enable the injection of bulking material between these tissues. In modified embodiments, the needle may further be placed into the layers of muscle of the lower esophageal sphincter to facilitate the injection of bulking agent into these tissues as well. In urethral procedures, the needle preferably penetrates through the mucosa but not through the muscle layers of the urinary sphincter, to thereby facilitate the injection of bulking material between these tissues; and in modified embodiments, the needle is further inserted into the layers of muscle of the urinary sphincter to facilitate the injection of bulking agent into these tissues as well. Uses of the injection facilitation apparatus 17 are not limited to the above examples; the invention encompasses other foreseeable uses such as injections of viscous or other materials through elongate catheters into the colon, vagina, vessels, and other lumen structures.

As illustrated above, uses of the injection facilitation apparatus 17 include the delivery of compositions into and/or adjacent to a sphincter muscle. Other sphincter muscles that are suitable target sites for delivery of compositions by said injection facilitation apparatus 17 include, but are not limited to, a pyloric sphincter, a bladder sphincter or an anal sphincter. In an embodiment, delivery of filler material using the injection facilitation apparatus 17 into, adjacent to, or into and adjacent to a pyloric sphincter is used to treat, for example, obesity. In another embodiment, delivery of filler material using the injection facilitation apparatus 17 into, adjacent to, or into and adjacent to a bladder sphincter is used to treat, for example, bladder incontinence. In another embodiment, delivery of filler material using the injection facilitation apparatus 17 into, adjacent to, or into and adjacent to an anal sphincter is used to treat, for example, fecal incontinence.

The injection facilitation apparatus 17 can also be used for embolization. Embolization or embolotherapy is the selective blockage of one or more blood vessels supplying a diseased vascular structure or diseased tissue while simultaneously preserving the blood supply to surrounding normal vascular structure or tissue. The injection facilitation apparatus 17 can be used to deliver filler composition to a site to accomplish a selective blockage. Embolization can be used as an adjunct to chemotherapy or radiation therapy. Embolization can be used as a treatment for cases of aneurysms, arteriovenous fistulae (AVFs), arteriovenous malformation (AVMs), and traumatic bleeding.

Medical conditions treated by using embolotherapy can be for vascular anomalies, such as AVM, AVF, venous malformation (VM), lymphatic malformation (LM), and hemangioma. Embolotherapy can be used for hemorrhage, such as pseudoaneurysms and gastrointestinal (GI) tract, pelvic, posttraumatic, epistaxis, and hemoptysis bleeding. Embolotherapy can be used for tumors, varicoceles, and organ ablation.

Vascular anomalies are grouped into two categories: hemangiomas and vascular malformations. Vascular malformations are categorized further as high-flow lesions (AVM, AVF), low-flow lesions (capillary malformation, VM, LM), or combined vascular malformations. Embolotherapy with a variety of embolic materials is commonly used in the treatment of vascular anomalies.

Several types of hemorrhage can be treated with embolization. Examples include hemoptysis; epistaxis; and GI tract, postpartum pelvic, posttraumatic, and iatrogenic hemorrhage (such as postbiopsy or nephrostomy tube insertion).

Embolization can be used for many types of malignant tumors. Renal malignancy is the most common type of tumor treated with embolotherapy. Unresectable tumors can be made operable by means of embolotherapy. Tumor metastasis can be controlled by embolization. Embolization can be used for palliation to control pain and hematuria. Other reported malignancies in which embolotherapy has been used include pelvic malignancies and bone tumors. Hemorrhage resulting from malignancy or radiotherapy (for example, due to radiation cystitis) can be controlled by using embolotherapy.

Embolization can be used to treat uterine leiomyomata or fibroids, commonly found in women in their 30s and 40s. Transcatheter embolotherapy is a well-recognized therapeutic alternative to surgery with a high patient satisfaction rate and positive therapeutic results.

Tissue augmentation can be used to repair or reinforce cartilage tissue, such as in the joints. Joint cartilage that can be augmented include knee menisci, which include the medial meniscus, lateral meniscus, and discoid meniscus. Knee menisci are known to be involved in joint stability, load sharing and transmission, shock absorption, and nutrition and lubrication of the articular cartilage.

Tissue augmentation can be used to repair or reinforce spinal discs. Disc injury includes annular tears with herniation of the nucleus pulposus (soft disc herniation) and annular tear without herniation of the nucleus pulposus (ie, internal disc disruption). Methods for treating disc injuries is described in U.S. Patent Publication No. 2006/0074424, entitled “Method of Treating a Spinal Internal Disc Derangement”, herein incorporated by reference. Filler materials can be used in the restoration of diseased intervertebral discs by means of arthroscopic implantation of filler materials directly into the depleted nucleus pulposus. The prosthesis can match the mechanical properties of the tissue being restored.

The method for tissue restoration of intervertebral discs in a mammal, comprises the step of injecting a polymer into the depleted nucleus pulposus site using the injection apparatus of the invention. Typically, during an endoscopic discectomy or other percutaneous procedure, the degraded or injured nucleus pulposus is removed from the intervertebral discs by commercially available endoscopic discectomy equipment. First, a trocar is used to make a hole in the annulus. The endoscopic discectomy equipment is then inserted through the hole and the nucleus pulposus removed. Injection of filler material can be injected into the site through the pre-formed hole, using the disclosed injection apparatus for loading and delivery of the filler material into the space vacated by the nucleus pulposus.

Although an exemplary embodiment of the invention has been shown and described, many other changes, modifications and substitutions, in addition to those set forth in the above paragraphs, may be made by one having ordinary skill in the art without necessarily departing from the spirit and scope of this invention. All references provided in the instant disclosure are herein incorporated by reference.

What is claimed is:

1. A method for placing an injectable therapeutic filler material to a targeted treatment location comprising:
inserting an injection apparatus wherein, said injection apparatus comprises
a transition-bore needle apparatus, which comprises a proximal end, a distal end, and a lumen extending from the proximal end to the distal end, wherein a diameter of the proximal end of the transition-bore needle apparatus is greater than a diameter of the distal end of the transition-bore needle apparatus, wherein a proximal portion of the transition-bore needle apparatus comprises a first needle having a first diameter, and a distal portion of the transition-bore needle apparatus comprises a second needle having a second diameter, and wherein the proximal end of the first needle defines a surface that is nonperpendicularly oriented to the lumen of the transition-bore needle apparatus; and,
a surgical device suitable for insertion within a body lumen;
and,
delivering said injectable therapeutic filler material through said injection apparatus to said targeted treatment location.

2. The method of claim 1, wherein said injection apparatus is guided by an external imaging system.

3. The method of claim 2, wherein said external imaging system is radiography, fluoroscopy, computerized tomography, or ultrasound.

4. The method of claim 2, wherein said external imaging system includes the injection of an image contrast agent.

5. The method of claim 1, wherein said body lumen is an esophagus, a stomach or duodenum.

6. The method of claim 5, wherein said surgical device is a gastroscope.

7. The method of claim 1, wherein said body lumen is a urethra or ureter.

8. The method of claim 7, wherein said surgical device is a cytoscope.

9. The method of claim 1, wherein said body lumen is a vagina or fallopian tube.

10. The method of claim 9, wherein said surgical device is a laproscope.

11. The method of claim 1, wherein said body lumen is a rectum, colon, large intestine or small intestine.

12. The method of claim 11, wherein said surgical device is a proctoscope or endoscope.

13. The method of claim 1, wherein said body lumen is a blood vessel or duct.

14. The method of claim 13, wherein said surgical device is angioscope.

15. The method of claim 1, wherein said body lumen is a bronchus or lung.

16. The method of claim 15, wherein said surgical device is a bronchoscope.

17. The method of claim 1, wherein said body lumen is a nasal sinus.

18. The method of claim 17, wherein said surgical device is an otoscope.

19. The method of claim 1, wherein said body lumen is a joint.

20. The method of claim 19, wherein said surgical device is an arthroscope.

21. The method of claim 1, wherein said surgical device comprises a liquid crystal polymer.

22. The method of claim 1, wherein said injectable therapeutic filler material is injected into or adjacent to an anatomic structure.

23. The method of claim 22, wherein said anatomic structure is a sphincter muscle.

24. The method of claim 23, wherein said sphincter muscle is an esophageal sphincter, a urinary sphincter, a pyloric sphincter, an anal sphincter, or a bladder sphincter.

25. The method of claim 1, wherein said injectable therapeutic filler material is soft tissue filler.

26. The method of claim 25, wherein said soft tissue filler comprises polymethylmethacrylate microspheres.

27. The method of claim 26, wherein said soft tissue filler comprises approximately 20% by weight polymethylmethacrylate microspheres and approximately 80% by weight of a composition comprising 3.5% purified bovine collagen, 2.7% phosphate buffer, 0.9% sodium chloride, 0.3% lidocaine hydrochloride, and 92.6% water for injection.

28. The method of claim 1, further comprising the step of mixing a composition of at least one cell type with said filler material.

29. The method of claim 28, wherein said cells are autogenic.

30. The method of claim 28, wherein said cells are human.

31. The method of claim 28, wherein said cells are genetically engineered.

32. The method of claim 28, wherein said cell type acts synergistically with other cell types in the formation of tissue.

33. The method of claim 28, wherein said cell type is selected from the group consisting of fibroblast cells, smooth muscle cells, striated muscle cells, heart muscle cells, nerve cells, epithelial cells, endothelial cells, bone cells, bone progenitor cells, bone marrow cells, blood cells, brain cells, kidney cells, liver cells, lung cells, pancreatic cells, spleen cells, breast cells, foreskin cells, ovary cells, testes cells and prostate cells.

34. The method of claim 28, wherein said cells are fetal or adults stem cells.

35. The method of claim 34, wherein said stem cells are totipotent, multipotent, or pluripotent.

36. The method of claim 1, wherein said filler material is mixed with a composition of at least one of non-mammalian eukaryotic cells, prokaryotic cells or viruses.

37. The method of claim 1, wherein said filler material is mixed with at least one of a physiologically buffered salt solution, water, or glycerol.

38. The method of claim 1, wherein said filler material is mixed with at least one composition of serum, a growth factor, a hormone, a sugar, an amino acid, a vitamin, a metalloprotein, or a lipoprotein.

39. The method of claim 28, wherein said targeted treatment location is selected from the group consisting of a blood vessel, a duct, a bronchus, a lung, a sinus, an esophagus, a stomach, a duodenum, a small intestine, a large intestine, a colon, a rectum, a ureter, a urethra, a vagina, a fallopian tube, a sphincter muscle, a spine and a joint

40. A method for treating gastro-esophogeal reflux disease comprising:
inserting an injection apparatus into the esophagus of said subject, said injection apparatus comprising
a transition-bore needle apparatus, which comprises a proximal end, a distal end, and a lumen extending from the proximal end to the distal end, wherein a diameter of the proximal end of the transition-bore needle apparatus is greater than a diameter of the distal end of the transition-bore needle apparatus, wherein a proximal portion of the transition-bore needle apparatus comprises a first needle having a first diameter, and a distal portion of the transition-bore needle apparatus comprises a second needle having a second diameter, and wherein the proximal end of the first needle defines a surface that is nonperpendicularly oriented to the lumen of the transition-bore needle apparatus; and,
a gastroscope;
and,
delivering an injectable therapeutic filler material through said injection apparatus around the lower esophageal sphincter to alter the biomechanical characteristics of said sphincter surrounding tissues to alleviate gastro-esophageal reflux.

41. The method of claim 40, wherein said injectable therapeutic filler material is soft tissue filler.

42. The method of claim 41, wherein said soft tissue filler comprises polymethylmethacrylate microspheres.

43. The method of claim 42, wherein said soft tissue filler comprises approximately 20% by weight polymethylmethacrylate microspheres and approximately 80% by weight of a composition comprising 3.5% purified bovine collagen, 2.7% phosphate buffer, 0.9% sodium chloride, 0.3% lidocaine hydrochloride, and 92.6% water for injection.

44. The method of claim 40, further comprising the step of mixing a composition of at least one type of cell with said filler material.

45. The method of claim 40, wherein said filler material is mixed with at least one of a physiologically buffered salt solution, water, or glycerol.

46. The method of claim 40, wherein said filler material is mixed with at least one composition of serum, a growth factor, a hormone, a sugar, an amino acid, a vitamin, a metalloprotein, or a lipoprotein.

47. A method for treating stress urinary incontinence comprising:
inserting an injection apparatus into the urethra of said subject, said injection apparatus comprising
a transition-bore needle apparatus, which comprises a proximal end, a distal end, and a lumen extending from the proximal end to the distal end, wherein a diameter of the proximal end of the transition-bore needle apparatus is greater than a diameter of the distal end of the transition-bore needle apparatus, wherein a proximal portion of the transition-bore needle apparatus comprises a first needle having a first diameter, and a distal portion of the transition-bore needle apparatus comprises a second needle having a second diameter, and wherein the proximal end of the first needle defines a surface that is nonperpendicularly oriented to the lumen of the transition-bore needle apparatus; and,
a cytoscope;
and,
delivering a therapeutic filler material through said injection apparatus around the urinary sphincter to alter the biomechanical characteristics of said sphincter surrounding tissues to alleviate stress urinary incontinence.

48. The method of claim 47, wherein said injectable therapeutic filler material is soft tissue filler.

49. The method of claim 48, wherein said soft tissue filler comprises polymethylmethacrylate microspheres.

50. The method of claim 49, wherein said soft tissue filler comprises approximately 20% by weight polymethylmethacrylate microspheres and approximately 80% by weight of a composition comprising 3.5% purified bovine collagen, 2.7% phosphate buffer, 0.9% sodium chloride, 0.3% lidocaine hydrochloride, and 92.6% water for injection.

51. The method of claim 47, further comprising the step of mixing a composition of at least one type of cell with said filler material.

52. The method of claim 47, wherein said filler material is mixed with at least one of a physiologically buffered salt solution, water, or glycerol.

53. The method of claim 47, wherein said filler material is mixed with at least one composition of serum, a growth factor, a hormone, a sugar, an amino acid, a vitamin, a metalloprotein, or a lipoprotein.

54. A method for enabling contraception comprising:
inserting an injection apparatus into the fallopian tube of a subject, said injection apparatus comprising
a transition-bore needle apparatus, which comprises a proximal end, a distal end, and a lumen extending from the proximal end to the distal end, wherein a diameter of the proximal end of the transition-bore needle apparatus is greater than a diameter of the distal end of the transition-bore needle apparatus, wherein a proximal portion of the transition-bore needle apparatus comprises a first needle having a first diameter, and a distal portion of the transition-bore needle apparatus comprises a second needle having a second diameter, and wherein the proximal end of the first needle defines a surface that is nonperpendicularly oriented to the lumen of the transition-bore needle apparatus; and,
a laproscope;
and,
delivering an injectable therapeutic filler material through said injection apparatus into said fallopian tube to provide a barrier and prevent fertilization.

55. The method of claim 54, wherein said injectable therapeutic filler material is soft tissue filler.

56. The method of claim 55, wherein said soft tissue filler comprises polymethylmethacrylate microspheres.

57. The method of claim 56, wherein said soft tissue filler comprises approximately 20% by weight polymethylmethacrylate microspheres and approximately 80% by weight of a composition comprising 3.5% purified bovine collagen, 2.7% phosphate buffer, 0.9% sodium chloride, 0.3% lidocaine hydrochloride, and 92.6% water for injection.

58. The method of claim 54, further comprising the step of mixing a composition of at least one type of cell with said filler material.

59. The method of claim 54, wherein said filler material is mixed with at least one of a physiologically buffered salt solution, water, or glycerol.

60. The method of claim 54, wherein said filler material is mixed with at least one composition of serum, a growth factor, a hormone, a sugar, an amino acid, a vitamin, a metalloprotein, or a lipoprotein.

61. A method for enabling contraception comprising:
inserting an injection apparatus into the vas deferens of a subject, said injection apparatus comprising
a transition-bore needle apparatus, which comprises a proximal end, a distal end, and a lumen extending from the proximal end to the distal end, wherein a diameter of the proximal end of the transition-bore needle apparatus is greater than a diameter of the distal end of the transition-bore needle apparatus, wherein a proximal portion of the transition-bore needle apparatus comprises a first needle having a first diameter, and a distal portion of the transition-bore needle apparatus comprises a second needle having a second diameter, and wherein the proximal end of the first needle defines a surface that is nonperpendicularly oriented to the lumen of the transition-bore needle apparatus; and,
an angioscope;
and,
delivering an injectable therapeutic filler material through said injection apparatus into said vas deferns to provide a barrier and prevent sperm from entering the ejaculatory duct.

62. The method of claim 61, wherein said injectable therapeutic filler material is soft tissue filler.

63. The method of claim 62, wherein said soft tissue filler comprises polymethylmethacrylate microspheres.

64. The method of claim 63, wherein said soft tissue filler comprises approximately 20% by weight polymethylmethacrylate microspheres and approximately 80% by weight of a composition comprising 3.5% purified bovine collagen, 2.7% phosphate buffer, 0.9% sodium chloride, 0.3% lidocaine hydrochloride, and 92.6% water for injection.

65. The method of claim 64, further comprising the step of mixing a composition of at least one type of cell with said filler material.

66. The method of claim 65, wherein said filler material is mixed with at least one of a physiologically buffered salt solution, water, or glycerol.

67. The method of claim 65, wherein said filler material is mixed with at least one composition of serum, a growth factor, a hormone, a sugar, an amino acid, a vitamin, a metalloprotein, or a lipoprotein.

68. A method for treating bladder incontinence comprising:
inserting an injection apparatus into the bladder of said subject, said injection apparatus comprising
a transition-bore needle apparatus, which comprises a proximal end, a distal end, and a lumen extending from the proximal end to the distal end, wherein a diameter of the proximal end of the transition-bore needle apparatus is greater than a diameter of the distal end of the transition-bore needle apparatus, wherein a proximal portion of the transition-bore needle apparatus comprises a first needle having a first diameter, and a distal portion of the transition-bore needle apparatus comprises a second needle having a second diameter, and wherein the proximal end of the first needle defines a surface that is nonperpendicularly oriented to the lumen of the transition-bore needle apparatus; and,
a cytoscope;
and,
delivering an injectable therapeutic filler material through said injection apparatus around the bladder sphincter to alter the biomechanical characteristics of said sphincter surrounding tissues to alleviate bladder incontinence.

69. The method of claim 68, wherein said injectable therapeutic filler material is soft tissue filler.

70. The method of claim 69, wherein said soft tissue filler comprises polymethylmethacrylate microspheres.

71. The method of claim 70, wherein said soft tissue filler comprises approximately 20% by weight polymethylmethacrylate microspheres and approximately 80% by weight of a composition comprising 3.5% purified bovine collagen, 2.7% phosphate buffer, 0.9% sodium chloride, 0.3% lidocaine hydrochloride, and 92.6% water for injection.

72. The method of claim 68, further comprising the step of mixing a composition of at least one type of cell with said filler material.

73. The method of claim 68, wherein said filler material is mixed with at least one of a physiologically buffered salt solution, water, or glycerol.

74. The method of claim 68, wherein said filler material is mixed with at least one composition of serum, a growth factor, a hormone, a sugar, an amino acid, a vitamin, a metalloprotein, or a lipoprotein.

75. A method for treating fecal incontinence comprising:
inserting an injection apparatus into the rectum of said subject, said injection apparatus comprising
a transition-bore needle apparatus, which comprises a proximal end, a distal end, and a lumen extending from the proximal end to the distal end, wherein a diameter of the proximal end of the transition-bore needle apparatus is greater than a diameter of the distal end of the transition-bore needle apparatus, wherein a proximal portion of the transition-bore needle apparatus comprises a first needle having a first diameter, and a distal portion of the transition-bore needle apparatus comprises a second needle having a second diameter, and wherein the proximal end of the first needle defines a surface that is nonperpendicularly oriented to the lumen of the transition-bore needle apparatus; and,
a proctoscope;
and,
delivering an injectable therapeutic filler material through said injection apparatus around the anal sphincter to alter the biomechanical characteristics of said sphincter surrounding tissues to alleviate fecal incontinence.

76. The method of claim 75, wherein said injectable therapeutic filler material is soft tissue filler.

77. The method of claim 76, wherein said soft tissue filler comprises polymethylmethacrylate microspheres.

78. The method of claim 77, wherein said soft tissue filler comprises approximately 20% by weight polymethylmethacrylate microspheres and approximately 80% by weight of a composition comprising 3.5% purified bovine collagen, 2.7% phosphate buffer, 0.9% sodium chloride, 0.3% lidocaine hydrochloride, and 92.6% water for injection.

79. The method of claim 75, further comprising the step of mixing a composition of at least one type of cell with said filler material.

80. The method of claim 75, wherein said filler material is mixed with at least one of a physiologically buffered salt solution, water, or glycerol.

81. The method of claim 75, wherein said filler material is mixed with at least one composition of serum, a growth factor, a hormone, a sugar, an amino acid, a vitamin, a metalloprotein, or a lipoprotein.

82. A method for treating obesity comprising:
inserting an injection apparatus into the stomach of said subject, said injection apparatus comprising
a transition-bore needle apparatus, which comprises a proximal end, a distal end, and a lumen extending from the proximal end to the distal end, wherein a diameter of the proximal end of the transition-bore needle apparatus is greater than a diameter of the distal end of the transition-bore needle apparatus, wherein a proximal portion of the transition-bore needle apparatus comprises a first needle having a first diameter, and a distal portion of the transition-bore needle apparatus comprises a second needle having a second diameter, and wherein the proximal end of the first needle defines a surface that is nonperpendicularly oriented to the lumen of the transition-bore needle apparatus; and,
a gastroscope;
and,
delivering an injectable therapeutic filler material through said injection apparatus around the pyloric sphincter to alter the biomechanical characteristics of said sphincter surrounding tissues to restrict gastric emptying.

83. The method of claim 82, wherein said injectable therapeutic filler material is soft tissue filler.

84. The method of claim 83, wherein said soft tissue filler comprises polymethylmethacrylate microspheres.

85. The method of claim 84, wherein said soft tissue filler comprises approximately 20% by weight polymethylmethacrylate microspheres and approximately 80% by weight of a composition comprising 3.5% purified bovine collagen, 2.7% phosphate buffer, 0.9% sodium chloride, 0.3% lidocaine hydrochloride, and 92.6% water for injection.

86. The method of claim 82, further comprising the step of mixing a composition of at least one type of cell with said filler material.

87. The method of claim 82, wherein said filler material is mixed with at least one of a physiologically buffered salt solution, water, or glycerol.

88. The method of claim 82, wherein said filler material is mixed with at least one composition of serum, a growth factor, a hormone, a sugar, an amino acid, a vitamin, a metalloprotein, or a lipoprotein.

89. A method for treating joint cartilage damage comprising:
inserting an injection apparatus into the damaged joint of said subject, said injection apparatus comprising
a transition-bore needle apparatus, which comprises a proximal end, a distal end, and a lumen extending from the proximal end to the distal end, wherein a diameter of the proximal end of the transition-bore needle apparatus is greater than a diameter of the distal end of the transition-bore needle apparatus, wherein a proximal portion of the transition-bore needle apparatus comprises a first needle having a first diameter, and a distal portion of the transition-bore needle apparatus comprises a second needle having a second diameter, and wherein the proximal end of the first needle defines a surface that is nonperpendicularly oriented to the lumen of the transition-bore needle apparatus; and,
an arthroscope;
and,
delivering an injectable therapeutic filler material through said injection apparatus into the joint to alter the biomechanical characteristics of said joint cartilage surrounding tissues to alleviate joint damage.

90. The method of claim 89, wherein said injectable therapeutic filler material is soft tissue filler.

91. The method of claim 90, wherein said soft tissue filler comprises polymethylmethacrylate microspheres.

92. The method of claim 91, wherein said soft tissue filler comprises approximately 20% by weight polymethylmethacrylate microspheres and approximately 80% by weight of a composition comprising 3.5% purified bovine collagen, 2.7% phosphate buffer, 0.9% sodium chloride, 0.3% lidocaine hydrochloride, and 92.6% water for injection.

93. The method of claim 89, further comprising the step of mixing a composition of at least one type of cell with said filler material.

94. The method of claim 89, wherein said filler material is mixed with at least one of a physiologically buffered salt solution, water, or glycerol.

95. The method of claim 89, wherein said filler material is mixed with at least one composition of serum, a growth factor, a hormone, a sugar, an amino acid, a vitamin, a metalloprotein, or a lipoprotein.

96. A method for embolotherapy comprising:
inserting an injection apparatus into the blood vessel of said subject, said injection apparatus comprising
a transition-bore needle apparatus, which comprises a proximal end, a distal end, and a lumen extending from the proximal end to the distal end, wherein a diameter of the proximal end of the transition-bore needle apparatus is greater than a diameter of the distal end of the transition-bore needle apparatus, wherein a proximal portion of the transition-bore needle apparatus comprises a first needle having a first diameter, and a distal portion of the transition-bore needle apparatus comprises a second needle having a second diameter, and wherein the proximal end of the first needle defines a surface that is nonperpendicularly oriented to the lumen of the transition-bore needle apparatus; and,
an angioscope;
and,
delivering an injectable therapeutic filler material through said injection apparatus into said blood vessel to interrupt or diminish blood flow.

97. The method of claim 96, wherein said injectable therapeutic filler material is soft tissue filler.

98. The method of claim 97, wherein said soft tissue filler comprises polymethylmethacrylate microspheres.

99. The method of claim 98, wherein said soft tissue filler comprises approximately 20% by weight polymethylmethacrylate microspheres and approximately 80% by weight of a composition comprising 3.5% purified bovine collagen, 2.7% phosphate buffer, 0.9% sodium chloride, 0.3% lidocaine hydrochloride, and 92.6% water for injection.

100. The method of claim 96, further comprising the step of mixing a composition of at least one type of cell with said filler material.

101. The method of claim 96, wherein said filler material is mixed with at least one of a physiologically buffered salt solution, water, or glycerol.

102. The method of claim 96, wherein said filler material is mixed with at least one composition of serum, a growth factor, a hormone, a sugar, an amino acid, a vitamin, a metalloprotein, or a lipoprotein.

103. The method of claim 96, wherein said blood flow is to a tumor.

104. The method of claim 103, wherein said tumor is carcinogenic.

105. The method of claim 96, wherein said blood vessel has an aneurysm, arteriovenous fistulae (AVF), arteriovenous malformation (AVM), or traumatic bleeding.

106. The method of claim 96, wherein said blood vessel comprises a venous malformation (VM), lymphatic malformation (LM), or hemangioma.

107. The method of claim 96, wherein said blood vessel is hemorrhaging.

108. The method of claim 107, wherein said hemorrhaging is from a pseudoaneurysm.

109. The method of claim 107, wherein said hemorrhaging is from gastrointestinal (GI) tract, pelvic, posttraumatic, epistaxis, or hemoptysis bleeding.

110. The method of claim 96, wherein said blood vessel is a varicocele.

111. The method of claim 96, wherein said blood vessel feeds an organ.

112. The method of claim 96, wherein said blood vessel feeds a uterine leiomyomata or fibroid.

113. A method for treating spinal disc injury comprising:
inserting an injection apparatus into the nucleus pulposus, or space vacated from the removal thereof, of said subject, said injection apparatus comprising
a transition-bore needle apparatus, which comprises a proximal end, a distal end, and a lumen extending from the proximal end to the distal end, wherein a diameter of the proximal end of the transition-bore needle apparatus is greater than a diameter of the distal end of the transition-bore needle apparatus, wherein a proximal portion of the transition-bore needle apparatus comprises a first needle having a first diameter, and a distal portion of the transition-bore needle apparatus comprises a second needle having a second diameter, and wherein the proximal end of the first needle defines a surface that is nonperpendicularly oriented to the lumen of the transition-bore needle apparatus; and,
endoscopic discectomy equipment;
and,
delivering an injectable therapeutic filler material through said injection apparatus into the nucleus pulposus, or space vacated from the removal thereof, to restore the biomechanical characteristics of said spinal surrounding tissues.

114. The method of claim 113, wherein said injectable therapeutic filler material is soft tissue filler.

115. The method of claim 114, wherein said soft tissue filler comprises polymethylmethacrylate microspheres.

116. The method of claim 115, wherein said soft tissue filler comprises approximately 20% by weight polymethylmethacrylate microspheres and approximately 80% by weight of a composition comprising 3.5% purified bovine collagen, 2.7% phosphate buffer, 0.9% sodium chloride, 0.3% lidocaine hydrochloride, and 92.6% water for injection.

117. The method of claim 113, further comprising the step of mixing a composition of at least one type of cell with said filler material.

118. The method of claim 113, wherein said filler material is mixed with at least one of a physiologically buffered salt solution, water, or glycerol.

119. The method of claim 113, wherein said filler material is mixed with at least one composition of serum, a growth factor, a hormone, a sugar, an amino acid, a vitamin, a metalloprotein, or a lipoprotein.

120. The method of claim 1, wherein said distal needle includes a depth indicator.
